(19) United States
(12) Patent Application Publication
(10) Pub. No.: US 2013/0052190 A1
(43) Pub. Date: Feb. 28, 2013

(54) CRTH2 Antagonists for Treatment of Eosinophilic Diseases and Conditions

(71) Applicants: Lisa Pearce Collins, Oxford (GB); Colin Michael Perkins, Oxford (GB); Michael Hunter, Bucks (GB); Eric Roy Pettipher, Faringdon (GB)

(72) Inventors: Lisa Pearce Collins, Oxford (GB); Colin Michael Perkins, Oxford (GB); Michael Hunter, Bucks (GB); Eric Roy Pettipher, Faringdon (GB)

(73) Assignee: Oxagen Limited, Abingdon (GB)

(21) Appl. No.: 13/402,582

(22) Filed: Feb. 22, 2012

Related U.S. Application Data
(60) Provisional application No. 61/445,407, filed on Feb. 22, 2011.

(51) Int. Cl. A61K 31/405; A61K 31/498; A61K 31/454; A61K 31/4439; A61K 31/47; A61K 31/44; A61K 31/4704; A61K 31/573; A61K 31/58; A61K 31/569; A61K 31/439; A61K 39/395; A61P 11/00; A61P 11/06; A61P 1/04; A61P 17/00; A61P 27/14; A61P 37/08; A61K 31/4709

(52) U.S. Cl. 424/133.1; 514/415; 514/314; 514/249; 514/323; 514/414; 514/339; 514/311; 514/351; 514/312; 514/171; 514/304; 514/291; 424/158.1

(57) ABSTRACT

The present invention provides a method for the treatment of allergic and inflammatory diseases or conditions by administering a compound of Formula (I). The invention provides a method of treatment that is particularly suited for patients with a high degree of airway eosinophilia in contrast to those with a lower degree of airway eosinophilia. The invention also provides a method of treatment that is particularly suited for patients with a high atopic status in contrast to those patients with a lower atopic status.

### BACKGROUND OF THE INVENTION

1. Field of the Invention

The present invention provides a method for the treatment of eosinophilic diseases or conditions by administering a compound of Formula (I). In particular, the invention provides a method of treatment that is particularly suited for patients with a high degree of airway eosinophilia in contrast to those with a lower degree of airway eosinophilia. The invention also provides a method of treatment that is particularly suited for patients with a high atopic status in contrast to those with a lower atopic status.

2. Related Art

Allergic conditions are becoming more common throughout the developed world with as much as 10% of the world's population being affected by one or more of allergic asthma, allergic rhinitis, atopic dermatitis and other allergic conditions. Numerous classes of compound have been suggested for the treatment of these conditions, with one of the more recent developments being the use of CRTH2 antagonists, which inhibit the action of agonists such as prostaglandin D₂ (PGD₂) acting at the CRTH2 receptor.

In patients who are affected by an allergic condition, the presence of an allergen to which the patient is sensitive induces the production of allergen-specific IgE antibodies by B cells, which reach the mature state in which they produce IgE with assistance from T cells, primarily Th2 cells. The allergen-specific IgE is present in the circulation and is also expressed on the surfaces of mast cells and basophils, which release inflammatory substances such as histamine, prostaglandin D₂ and tryptase when the allergen binds to the IgE.

Thus, allergy can be characterised by an increase in atopy, which involves an increase in the number of cells reactive to allergens present in the circulation and tissues of a patient and which may lead to the presence in the circulation and on the surfaces of mast cells and basophils of IgE specific for an allergen to which a patient is sensitive.

PGD₂ is an eicosanoid, a class of chemical mediator synthesised by cells in response to local tissue damage, normal stimuli or hormonal stimuli or via cellular activation pathways. Eicosanoids bind to specific cell surface receptors on a wide variety of tissues throughout the body and mediate various effects in these tissues. PGD₂ is known to be produced by mast cells, macrophages and Th2 lymphocytes and has been detected in high concentrations in the airways of asthmatic patients challenged with antigen (Murray et al, (1986), N. Engl. J. Med. 315:800-804). Instillation of PGD₂ into airways can provoke many features of the asthmatic response including bronchoconstriction (Hardy et al, (1984) N. Engl. J. Med. 311:209-213; Sampson et al, (1997) Thorax 52:513-518) and eosinophil accumulation (Emery et al, (1989) J. Appl. Physiol 67:959-962).

The potential of PGD₂ to induce inflammatory responses has been confirmed by the use of transgenic mice overexpressing human PGD₂ synthase which exhibit exaggerated eosinophilic lung inflammation and Th2 cytokine production in response to antigen (Fujitani et al, (2002) J. Immunol. 168:443-449).

The first receptor specific for PGD₂ to be discovered was the DP₁ receptor which is linked to elevation of the intracellular levels of cAMP. However, PGD₂ is thought to mediate much of its proinflammatory activity through interaction with a G protein-coupled receptor termed CRTH2 (chemoattractant receptor-homologous molecule expressed on Th2 cells) which is expressed by Th2 lymphocytes, eosinophils and basophils (Hirai et al, (2001) J. Exp. Med. 193:255-261, and EP0851030 and EP-A-1211513 and Bauer et al, EP-A-1170594). It seems clear that the effect of PGD₂ on the activation of Th2 lymphocytes and eosinophils is mediated through CRTH2 since the selective CRTH2 agonists 13,14 dihydro-15-keto-PGD₂ (DK-PGD₂) and 15R-methyl-PGD₂ can elicit this response and the effects of PGD₂ are blocked by an anti-CRTH2 antibody (Hirai et al, 2001; Monneret et al, (2003) J. Pharmacol. Exp. Ther. 304:349-355). In contrast, the selective DP agonist BW245C does not promote migration of Th2 lymphocytes or eosinophils (Hirai et al, 2001; Gervais et al, (2001) J. Allergy Clin. Immunol. 108:982-988). Based on this evidence, antagonising PGD₂ at the CRTH2 receptor is an attractive approach to treat the inflammatory component of Th2-dependent allergic diseases such as asthma (including allergic asthma), food allergies, acute and chronic urticaria, perennial allergic rhinitis, seasonal allergic rhinitis, atopic dermatitis, contact hypersensitivity (including contact dermatitis) and conjunctivitis, especially allergic conjunctivitis.

Documents which discuss the use of CRTH2 antagonists for the treatment of Th2-dependent allergic diseases include WO-A-03/066046, WO-A-03/066047, WO-A-03/097042, WO-A-03/097598, WO-A-03/101981, WO-A-03/101961, WO-A-2004/007451, WO-A-2005/019171, WO-A-2005/094816, WO-A-2005/044260, WO-A-2005/040112, WO-A-2005/040114, WO2006/095183, and WO2008/012511.

Other compounds which are combined CRTH2 and TP receptor antagonists are known and an example of such a compound is ramatroban, which has been shown to reduce allergic inflammation in the guinea pig nasal mucosa (Narita et al., (1996), Int. Arch. Allergy Immunol. 109:161-166), mouse airways (Nagai et al, (1995), Prostaglandins 50:75-87) and mouse skin (Takeshita et al., (2004), Int. Immunol. 16:947-959). Ramatroban has also shown to reduce symptoms of perennial allergic rhinitis in human subjects (Terada et al., (1998), Allergol. Int. 47: 59-67). Analogues of ramatroban that retain CRTH2 antagonist activity, but which are inactive on TP, are effective in reducing airway eosinophilia and mucus cell hyperplasia in a model of allergic asthma in mice (Uller et al., (2007), Respir. Res. 8:16). Uller et al. concluded that the efficacy in the allergic asthma model resulted from blockade of CRTH2 receptors rather than inhibition of TP.

In mouse models, persistent airway inflammation and bronchial hyper-responsiveness is dependent upon the production of Th2 cytokines (Schwarze et al., (2000), Thorax 59:517-521) and, interestingly, Th2 cytokines can induce ICAM-1 expression by bronchial epithelial cells (Bianco et al., (1998), Eur. Respir. J. 12:619-626). Conversely, the Th1 cytokine interferon-γ down-regulates ICAM-1 rhinovirus-induced expression by bronchial epithelial cells and promotes shedding of ICAM-1 from the surface of these cells (Whiteman and Spiteri, (2008), Journal of Inflammation 5:8) which is thought to play a protective role against infection with rhinovirus. Therefore, overproduction of Th2 cytokines not only leads to the manifestation of symptoms during viral exacerbations but may also predispose to infection with rhinovirus.

Indeed, a high Th2 to Th1 cytokine ratio in the airways is associated with more severe cold symptoms and delayed viral clearance in human subjects infected with rhinovirus (Gem et al., (2000), Am. J. Respir. Crit. Care Med. 62:2226-2231).

Th2 inflammation is believed to be the central molecular mechanism underlying asthma with two distinct phenotypes based on the degree of Th2 inflammation (Woodruff et al., (2009), Am. J. Resp. Crit. Care Med. 180:388-395). Measurement of expression levels of Th2 cytokine genes (IL-5 and IL-13) in bronchial biopsies utilizing gene arrays revealed two distinct phenotypes, “Th2-high” asthma and “Th2-low” asthma. The two subgroups differed in their expression of IL-5 and IL-13 in bronchial biopsies and airway hyper-responsiveness, serum IgE, blood and airway eosinophilia, subepithelial fibrosis, and airway mucin gene expression.

A Th2 dominant form of asthma is characterized by eosinophilic airway inflammation which can be defined as sputum eosinophilia >2% or >3% but other measurements can serve as surrogates e.g. blood eosinophilia >250/ul, exhaled NO>50 ppb or the presence of a “Th2 high” genomic signature in cells derived from the airways of asthmatic subjects. The Th2 high genomic signature is defined as elevated expression of genes for periostin (POSTN), chloride channel regulator 1 (CLCA1) and serpin peptidase inhibitor, clade B, member 2 (SERPINB2).

Since CRTH2 plays a pivotal role in Th2 cytokine production during allergic responses, the inventors postulated that CRTH2 antagonists may be effective in treating or preventing eosinophilic diseases and conditions in patients with a high degree of airway eosinophilia or atopy. Therefore, the inventors carried out a study of patients with asthma and found that patients with a high degree of airway eosinophilia or atopy, as determined by FEV₁ profile, eosinophil count, and skin prick test, showed greater improvement after treatment with a CRTH2 antagonist compared to patients with a lower degree of airway eosinophilia or atopy.

### BRIEF SUMMARY OF THE INVENTION

One aspect of the invention is to provide a method of treating an eosinophilic disease or condition in a subject, comprising:
(a) determining the degree of airway eosinophilia in the subject;
(b) administering a compound of Formula (I) to said subject if the degree of airway eosinophilia in (a) is at or above a level determined to be high;
   wherein the compound of Formula (I):

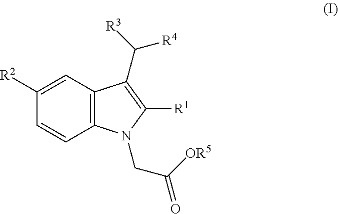

wherein
R¹ is C₁-C₆ alkyl;
R² is halogen; and
R³ is aryl or heteroaryl optionally substituted with one or more substituents selected from halo, OH, CN, R⁶, COR⁶, CH₂R⁶, OR⁶, SR⁶, SO₂R⁶ or SO₂YR⁶;
R⁶ is C₁-C₆ alkyl, C₃-C₈ cycloalkyl, heterocyclyl, aryl or heteroaryl, any of which may optionally be substituted with one or more substituents selected from halo, OH, CN, NO₂, C₁-C₆ alkyl or O(C₁-C₆ alkyl); and
Y is NH or a straight or branched C₁-C₄ alkylene chain;
   R⁴ is H or C₁-C₄ alkyl;
   R⁵ is hydrogen, C₁-C₆ alkyl, aryl, (CH₂)ₘOC(═O)C₁-C₆alkyl, ((CH₂)ₘO)ₙCH₂CH₂X, (CH—₂)ₘN(R⁷)₂ or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
   or a pharmaceutically acceptable salt, hydrate, solvate, or complex thereof.

In one embodiment, R⁵ of Formula (I) is hydrogen.

In one embodiment, R⁵ of Formula (I) is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

In one embodiment, R¹ of Formula (I) is C₁-C₄ alkyl; R² is fluoro; R³ is optionally substituted and is quinoline, quinoxaline, isoquinoline, thiazole, phenyl, naphthalene, thiophene, pyrrole, or pyridine; and R⁴ is H or methyl.

In one embodiment, R⁴ of Formula (I) is H.

In one embodiment, R³ of Formula (I) is optionally substituted and is quinoline, isoquinoline, phenyl, naphthalene, thiophene, pyrrole, or pyridine.

In one embodiment, R³ of Formula (I) is quinoline or isoquinoline, wherein the quinoline or isoquinoline is unsubstituted or substituted with one or more halo substituents.

In one embodiment, R³ of Formula (I) is optionally substituted with one or more substituents and is phenyl, naphthalene, thiophene, pyrrole, or pyridine, wherein the one or more substituents are OR⁶, SO₂R⁶ or SO₂YR⁶.

In one embodiment, R⁶ of Formula (I) is optionally substituted and is C₁-C₆ alkyl, a 4- to 6-membered cycloalkyl group, a 5- or 6-membered heterocyclyl group, or phenyl.

In one embodiment, R³ of Formula (0 is a 3-pyridyl moiety.

In one embodiment, R³ of Formula (I) is substituted with SO₂YR⁶, wherein Y is a CH₂ moiety.

In one embodiment, R³ of Formula (I) is substituted with SO₂R⁶ or SO₂YR⁶, wherein the R⁶ group is unsubstituted or substituted with one or more substituents selected from methyl and halo.

In one embodiment, R³ of Formula (I) is substituted with OR⁶, wherein the R⁶ group is unsubstituted or substituted with one or more substituents selected from the group consisting of halo, cyano, C₁-C₄ alkyl, and O(C₁-C₄ alkyl).

In one embodiment, the compound of Formula (I) is:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-((1-[(4-methylbenzene)sulfonyl)pyrrol-2-yl]methyl)indol-1-yl]-acetic acid;
[3-({1-(2,4-Difluorobenzene)sulfonyl]pyrrol-2-yl)methyl)-5-fluoro-2-methylindol-1-yl}-acetic acid;
(3-{[2-(Benzenesulfonyl)phenyl]methyl}-5-fluoro-2-methylindol-1-yl)-acetic acid;
[3-({(2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]acetic acid;
[3-({(2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
(5-Fluoro-3-[(1-methanesulfonylnaphthalen-2-yl)methyl]-2-methylindol-1-yl)-acetic acid;
(5-Fluoro-3-[(6-methanesulfonylnaphthalen-2-yl)methyl]-2-methylindol-1-yl)-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl)indol-1-yl}-acetic acid;
(5-Fluoro-2-methyl-3-{[3-(methylsulfanyl)phenyl]methyl}indol-1-yl)-acetic acid;
(5-Fluoro-2-methyl-3-{[4-(ethyl sulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-{[4-(n-propylsulfanyl)phenyl]methyl}indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-{[4-(pentan-3-ylsulfanyl)phenyl]methyl}indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-([2-(Ethanesulfonyl)phenyl]methyl)-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-([2-(Butane-2-sulfonyl)phenyl]methyl)-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-[4-(pentan-3-ylsulfonyl)phenyl]methyl)indol-1-yl)-acetic acid;
[3-({4-[(Cyclopropyl methyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-([2-(4-Chlorophenyl)phenyl]methyl)-5-fluoro-2-methylindol-1-yl)-acetic acid;
(5-Fluoro-2-methyl-3-{[2-(4-methyl phenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
{5-Fluoro-2-methyl-3-[(3-phenoxythiophen-2-yl)methyl]indol-1-yl}-acetic acid;
(3-{[2-(Benzenesulfonyl)-1,3-thiazol-5-yl]methyl}-5-fluoro-2-methylindol-1-yl)-acetic acid;
{3-[(4-Benzylpyrazol-4-yl)methyl]-5-fluoro-2-methylindol-1-yl}-acetic acid;
(3-([5-(4-Chlorophenoxy)-1-methyl-3-(trifluoromethyl)pyrazol-4-yl]methyl)-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
   or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
   m is 1 or 2;
   n is 1-4;
   X is OR⁷ or N(R⁷)₂;
   R⁷ is hydrogen or methyl; and
   R⁸ is C₁-C₁₈ alkyl.

In one embodiment, the compound of Formula (I) is administered in combination with one or more additional agent which is of use in the treatment of an eosinophilic disease or condition and/or which is useful in the treatment of an atopic disease or condition.

In one embodiment, the additional agent administered in combination with the compound of Formula (I) is selected from the group consisting of montelukast; β adrenoreceptor agonists such as metaproterenol, isoproterenol, isoprenaline, albuterol, salbutamol, formoterol, salmeterol, terbutaline, orciprenaline, bitolterol mesylate, pirbuterol, and indacaterol; inhaled corticosteroids such as prednisone, prednisolone, flunisolide, triamcinolone acetonide, beclomethasone dipropionate, budesonide, fluticasone propionate, fluticasone furoate, mometasone furoate, and ciclesonide; muscarinic receptor antagonists such as ipratropium and tiotropium; anti-IL-5 antibodies such as mepolizumab and reslizumab; anti-IgE antibodies such as omalizumab; and agents which block the actions of IL-4 and/or IL-13 such as the mutein pitrakinra and anti-IL-13 receptor α antibodies such as AMG-317 and anti-IL-13 antibodies.

In one embodiment, the compound of Formula (I) is administered via the oral, nasal, bronchial, or topical route.

In another embodiment, the present invention provides a method of treating or preventing an eosinophilic disease or condition, comprising administering to a subject in need of such treatment a compound of Formula (I), wherein the eosinophilic disease or condition is selected from the group consisting of eosinophilic asthma, atopic asthma, uncontrolled asthma, eosinophilic chronic obstructive pulmonary disease, eosinophilic nasal polyps, eosinophilic oesophagitis, eosinophilic atopic dermatitis, eosinophilic allergic conjunctivitis, allergic rhinitis, and Churg Strauss syndrome.

In one embodiment, the degree of airway eosinophilia is measured based on baseline blood eosinophil count. In one embodiment, the degree of airway eosinophilia is determined to be high when the baseline blood eosinophil count is greater than 200 cells/μL. In another embodiment, the degree of airway eosinophilia is determined to be high when the baseline blood eosinophil count is greater than 250 cells/μL.

In another embodiment, the degree of airway eosinophilia is measured based on baseline sputum eosinophil percentage. In one embodiment, the degree of airway eosinophilia is determined to be high when the baseline sputum eosinophil percentage is greater than 2%. In another embodiment, the degree of airway eosinophilia is determined to be high when the baseline sputum eosinophil percentage is greater than 2.5%. In another embodiment, the degree of airway eosinophilia is determined to be high when the baseline sputum eosinophil percentage is greater than 3%.

In another embodiment, the degree of airway eosinophilia is measured based on baseline ACQ (asthma control questionnaire) score. In one embodiment, the degree of airway eosinophilia is determined to be high when the baseline ACQ score is at least 1.5. In another embodiment, the degree of airway eosinophilia is determined to be high when the baseline ACQ score is at least 2.0.

In another embodiment, the degree of airway eosinophilia is measured based on baseline ACQ score and baseline blood eosinophil count. In one embodiment, the degree of airway eosinophilia is determined to be high when the baseline ACQ score is at least 1.5 and the baseline blood eosinophil count is greater than 200 cells/μL. In another embodiment, the degree of airway eosinophilia is determined to be high when the baseline ACQ score is at least 1.5 and the baseline blood eosinophil count is greater than 250 cells/μL.

In another embodiment, the present invention provides a method of treating an eosinophilic disease or condition in a subject, comprising:
(a) determining the degree of airway eosinophilia in the subject;
(b) administering a compound of Formula (I) to said subject if the degree of airway eosinophilia in (a) is at or above a level determined to be high;
   wherein the subject also has a high atopic status. In one embodiment, the atopic status is measured based on a skin prick test. In one embodiment, the atopic status is determined to be high when the skin prick test is positive and the degree of airway cosinophilia is determined to be high when the baseline ACQ score is at least 1.5 and the baseline blood eosinophil count is greater than 200 cells/μL.

In another embodiment, the present invention provides a method of treating or preventing an eosinophilic disease or condition, comprising administering to a subject in need of such treatment a compound of Formula (I), wherein the subject is less than or equal to 50 years of age. In another embodiment, the subject is less than or equal to 30 years of age.

In another embodiment, the present invention provides a method of treating or preventing an eosinophilic disease or condition, comprising administering to a subject in need of such treatment a compound of Formula (I), wherein the subject is less than or equal to 30 years of age at onset of the eosinophilic disease or condition. In another embodiment, the subject is between about 11 and about 20 years of age at onset of the eosinophilic disease or condition. In another embodiment, the subject is between about 0 and about 10 years of age at onset of the eosinophilic disease or condition.

In another embodiment, the present invention provides a method of treating an eosinophilic disease or condition in a subject, comprising:
(a) determining the atopic status of the subject;
(b) administering a compound of Formula (I) to said subject if the atopic status in (a) is at or above a level determined to be high.

In one embodiment, the atopic status is measured based on serum IgE levels. In another embodiment, the atopic status is determined to be high when the serum IgE level is greater than 100 units/mL. In another embodiment, the atopic status is determined to be high when the serum IgE level is greater than 200 units/mL.

### BRIEF DESCRIPTION OF DRAWINGS

FIG. 1 provides a graph of the change in FEV₁ over twelve weeks of treatment for patients with a FEV₁ measurement of 60-80% of predicted and a positive skin prick test comparing patients taking (5-fluoro-2-methyl-3-quinolin-2-ylmethyl-indol-1-yl)-acetic acid (OC000459) and patients taking a placebo.

FIG. 2 provides a graph of the change in FEV₁ over twelve weeks of treatment for patients with a FEV₁ measurement of 60-80% of predicted, a positive skin prick test, and a blood eosinophil count of greater than or equal to 250/μL comparing patients taking OC000459 and patients taking a placebo.

FIG. 3 provides a graph of the change in FEV₁ over twelve weeks of treatment for patients with a FEV₁ measurement of 60-80% of predicted, a positive skin prick test, and a blood eosinophil count of less than 250/μL comparing patients taking OC000459 and patients taking a placebo.

FIG. 4 provides a graph of the change in FEV₁ over twelve weeks of treatment for patients with an ACQ (asthma control questionnaire) score greater than or equal to 1.5 at baseline comparing patients taking OC000459 and patients taking a placebo.

FIG. 5 provides a graph of the change in FEV₁ over twelve weeks of treatment for patients with an ACQ (asthma control questionnaire) score less than 1.5 at baseline comparing patients taking OC000459 and patients taking a placebo.

FIG. 6 provides a graphical comparison of change in FEV₁ over four weeks of treatment for patients taking OC000459 and patients taking placebo for (a) full population of patients, (b) patients with serum eosinophil levels of ≧250/μL at baseline, and (c) patients with serum eosinophil levels of <250/μL at baseline.

FIG. 7 provides a Kaplan-Meier plot comparing the percent of patients with an asthma exacerbation over 20 weeks for patients taking OC000459 and patients taking placebo for (a) patients with serum eosinophil levels of ≧250/μL at baseline and (b) patients with serum eosinophil levels of <250/μL at baseline.

### DETAILED DESCRIPTION OF THE INVENTION

Unless otherwise defined, all technical and scientific terms used herein have the meaning commonly understood by one skilled in the art to which this invention belongs. All publications, patent applications, patents, and other references mentioned herein are incorporated in reference in their entirety. In case of conflict, the present specification, including definitions, will control.

The term “subject,” as used herein refers to an animal, preferably a mammal, most preferably a human, who is the object of treatment.

Although there is evidence that CRTH2 antagonists are effective in treating allergic asthma, allergic rhinitis, allergic conjunctivitis, atopic dermatitis, and other allergic conditions, the inventors have made the surprising discovery that subjects with a high degree of airway eosinophilia show a greater improvement after administration of a compound of Formula (I) compared to subjects with a lower degree of airway eosinophilia.

The inventors have also discovered that subjects with a high atopic status show a greater improvement after administration of a compound of Formula (I) compared to subjects with a lower atopic status.

The term “atopic status” as used herein may be characterized by the presence in the circulation or tissues of a patient of cells reactive to allergens, for example, mast cells, basophils, Th2 cells, dendritic cells, and B cells. A patient with a high atopic status may be characterized by the presence in the circulation or tissues of an increased number of cells reactive to allergens as compared to the general population. A further characteristic of patients with atopy may be the presence of IgE which binds specifically to an allergen to which the patient is sensitive. The IgE may be circulating or bound, for example, to the surface of mast cells and basophils. In particular, the patient may have a Th1/Th2 imbalance, such that the ratio of Th2 to Th1 cells is higher than that found in the general population.

In one embodiment, the invention provides a method of treating an eosinophilic disease in a subject with a high degree of airway eosinophilia comprising administering a compound of Formula (I). The degree of airway eosinophilia in a subject can be determined by any method commonly used by one of skill in the art.

In another embodiment, the invention provides a method of treating an eosinophilic disease in a subject with a high atopic status. The atopic status of a subject can be determined by any method commonly used by one of skill in the art.

In one embodiment, the degree of a subject's response to treatment with a compound of Formula (I) can be based on established standards.

According to the National Asthma Education and Prevention Program Expert Panel Report (Expert Panel Report 3: Guidelines for the Diagnosis and Management of Asthma, 2007, U.S. Department of Health and Human Services), asthma severity can be classified into four types based on four main factors: nighttime awakenings, short-acting β₂-agonist use for symptom control, interference with normal activity, and lung function. Overall asthma severity is based on the worst individual variable.

According to the Method of Expert Panel Report, type 1 asthma is termed intermittent asthma. With intermittent asthma, symptoms occur usually less than two days per week with a forced expiratory volume (FEV₁) measurement greater than 80% predicted. Oral corticosteroids are usually not required for intermittent asthma.

In mild asthma (type 2 according to the Method of Expert Panel Report), symptoms occur more than 2 days per week but not daily with a FEV₁ measurement ≧80% predicted. For mild asthma, the recommended medication is treatment with low-dose inhaled corticosteroids.

In moderate asthma (type 3 according to the Method of Expert Panel Report), symptoms occur daily with a FEV₁ greater than 60% but less than 80% predicted. For moderate asthma administration of low to medium dose corticosteroids in combination with long-acting inhaled β₂ agonists is recommended.

Finally, severe asthma (type 4 according to the Method of Expert Panel Report) symptoms occur throughout the day with a FEV₁ less than 60% predicted. Severe asthma is usually treated with high-dose inhaled corticosteroids in combination with long acting inhaled β₂ agonists plus one or more of the following if needed: sustained release theophylline, leukotriene modifier, long-acting oral β₂ agonists, and oral corticosteroids.

In one embodiment, the invention provides a method of treating or preventing asthma in a subject with a high degree of airway eosinophilia and/or a high atopic status comprising administering a compound of Formula (I). In another embodiment, the invention provides a method of treating or preventing asthma in a subject with a high degree of airway eosinophilia and/or a high atopic status comprising administering a pharmaceutical composition comprising a compound of Formula (I).

According to the Global Initiative for Chronic Obstructive Lung Disease (Pocket Guide to COPD Diagnosis, Management and Prevention, A Guide for Health Care Professionals, updated 2009), the are four stages of chronic obstructive pulmonary disease (COPD).

During stage I, termed mild COPD, there may be mild airflow limitation with a FEV₁≧80% predicted. During stage II, termed moderate COPD, there may be a worsening of the airflow limitation with a FEV₁ of less than 80% predicted. It is at this stage that people typically seek medical treatment. During stage III, termed severe COPD, there is a further worsening of the airflow limitation with FEV₁ of less than 50% predicted. During stage IV, termed very severe COPD, there is severe airflow limitation with a FEV₁ of less than 30% predicted.

In one embodiment, the invention provides a method of treating or preventing COPD in a subject with a high degree of airway cosinophilia and/or a high atopic status comprising administering a compound of Formula (I). In another embodiment, the invention provides a method of treating or preventing COPD in a subject with a high degree of airway eosinophilia and/or a high atopic status comprising administering a pharmaceutical composition comprising a compound of Formula (I).

In one embodiment, the degree of airway eosinophilia or atopic status of the subject can be measured by factors including, but not limited to, positive skin prick test, asthma control questionnaire (ACQ) score, baseline FEV₁ measurement, baseline blood eosinophil count, baseline sputum eosinophil percentage, fractional exhaled nitric oxide (FE_{NO}) level, and immunoglobulin E (IgE) levels.

In one embodiment, the atopic status of a subject is determined by a positive skin prick test. A skin prick test may be performed using any method known to one of skill in the art. Skin prick positivity is shown by one or more positive skin prick tests.

In one embodiment, the degree of airway eosinophilia is determined by blood eosinophil count. Blood eosinophil count may be measured by any method known to one of skill in the art. In one embodiment, the blood eosinophil count is measured by histology. In another embodiment, the blood eosinophil count is measured by flow cytometry.

In one embodiment, a subject has a blood eosinophil count of between 200 and 1000 cells/pt prior to the administration of the one or more doses of a compound of Formula (I). In another embodiment, a subject has a blood eosinophil count between 250 and 1000 cells/μL, between 300 and 1000 cells/μL, between 400 and 1000 cells/μL, between 500 and 1000 cells/μL, or between 200 and 500 cells/μL prior to the administration of the one or more doses of a compound of Formula (I). In another embodiment, a subject has a blood eosinophil count of at least 200 cells/μL, at least 250 cells/μL, at least 300 cells/μL, at least 400 cells/μL, or at least 500 cells/μL prior to the administration of the one or more doses of a compound of Formula (I). In another embodiment, a subject has a blood eosinophil count of 200 cells/μL prior to the administration of the one or more doses of a compound of Formula (I). In another embodiment, a subject has a blood eosinophil count of 250 cells/μL prior to the administration of the one or more doses of a compound of Formula (I). In another embodiment, a subject has a blood eosinophil count of 300 cells/μL prior to the administration of the one or more doses of a compound of Formula (I). In another embodiment, a subject has a blood eosinophil count of 350 cells/μL prior to the administration of the one or more doses of a compound of Formula (I). In another embodiment, a subject has a blood eosinophil count of 400 cells/μL prior to the administration of the one or more doses of a compound of Formula (I). In another embodiment, a subject has a blood eosinophil count of 500 cells/μL prior to the administration of the one or more doses of a compound of Formula (I).

In another embodiment, the degree of airway eosinophilia is determined by percentage eosinophil in induced sputum, i.e. the number of eosinophils expressed as a percentage of the total number of cells in a sputum sample. The percentage eosinophil in induced sputum may be measured using any method known to one of ordinary skill in the art. For example, methods of measuring percentage cosinophil in induced sputum are described in Belda et al., (2000), Am. J. Resp. Crit. Care Med 161:475-478.

In one embodiment, a subject has a % eosinophil in induced sputum of between 2% and 10% prior to the administration of the one or more doses of a compound of Formula (I). In another embodiment, a subject has a % eosinophil in induced sputum between 2% and 5%, between 3% and 5%, between 3% and 10%, or between 2.5% and 5%, prior to the administration of the one or more doses of a compound of Formula (I). In another embodiment, a subject has a % eosinophil in induced sputum of at least 2%, at least 2.5%, at least 3%, at least 4%, at least 5%, at least 6%, at least 7%, at least 8%, at least 9%, or at least 10% prior to the administration of the one or more doses of a compound of Formula (I). In another embodiment, a subject has a % eosinophil in induced sputum of 2% prior to the administration of the one or more doses of a compound of Formula (I). In another embodiment, a subject has a % eosinophil in induced sputum of 2.5% prior to the administration of the one or more doses of a compound of Formula (I). In another embodiment, a subject has a % eosinophil in induced sputum of 3% prior to the administration of the one or more doses of a compound of Formula (I). In another embodiment, a subject has a % eosinophil in induced sputum of 4% prior to the administration of the one or more doses of a compound of Formula (I). In another embodiment, a subject has a % eosinophil in induced sputum of 5% prior to the administration of the one or more doses of a compound of Formula (I). In another embodiment, a subject has a % eosinophil in induced sputum of 6% prior to the administration of the one or more doses of a compound of Formula (I). In another embodiment, a subject has a %, eosinophil in induced sputum of 7% prior to the administration of the one or more doses of a compound of Formula (I). In another embodiment, a subject has a % eosinophil in induced sputum of 8% prior to the administration of the one or more doses of a compound of Formula (I). In another embodiment, a subject has a % eosinophil in induced sputum of 9% prior to the administration of the one or more doses of a compound of Formula (I). In another embodiment, a subject has a % eosinophil in induced sputum of 10% prior to the administration of the one or more doses of a compound of Formula (I).

In one embodiment, the degree of airway cosinophilia is determined by fractional exhaled nitric oxide (FE_{NO}). FE_{NO} may be measured using any method known to one of ordinary skill in the art. FE_{NO} measurements are quick and easy to perform and have been found to be a good diagnostic tool for asthma (Smith et al., (2004), Am. J. Resp. Crit. Care Med. 169:473-478).

In one embodiment, a subject has a FE_{NO} level of between 50 and 300 ppb (parts per billion) prior to the administration of the one or more doses of a compound of Formula (I). In another embodiment, a subject has a FE_{NO} level of between 60 and 300 ppb, between 70 and 300 ppb, between 80 and 300 ppb, between 90 and 300 ppb, between 100 and 300 ppb, between 110 and 300 ppb, between 120 and 300 ppb, between 130 and 300 ppb, or between 150 and 300 ppb prior to the administration of the one or more doses of a compound of Formula (I). In another embodiment, a subject has a FE_{NO} level of at least 50 ppb, at least 60 ppb, at least 70 ppb, or at least 100 ppb prior to the administration of the one or more doses of a compound of Formula (I). In another embodiment, a subject has a FE_{NO} level of 50 ppb prior to the administration of a compound of Formula (I). In another embodiment, a subject has a FE_{NO} level of 60 ppb prior to the administration of the one or more doses of a compound of Formula (I). In another embodiment, a subject has a FE_{NO} level of 70 ppb prior to the administration of the one or more doses of a compound of Formula (I). In another embodiment, a subject has a FE_{NO} level of 80 ppb prior to the administration of the one or more doses of a compound of Formula (I). In another embodiment, a subject has a FE_{NO} level of 90 ppb prior to the administration of the one or more doses of a compound of Formula (I). In another embodiment, a subject has a FE_{NO} level of 100 ppb prior to the administration of the one or more doses of a compound of Formula (I). In another embodiment, a subject has a FE_{NO} level of 110 ppb prior to the administration of the one or more doses of a compound of Formula (I). In another embodiment, a subject has a FE_{NO} level of 120 ppb prior to the administration of the one or more doses of a compound of Formula (I). In another embodiment, a subject has a FE_{NO} level of 130 ppb prior to the administration of the one or more doses of a compound of Formula (I).

The asthma control questionnaire (ACQ) score (Juniper et al, (1999) Eur Respir. J. 14:902-907) is a well-known and internationally accepted method for determining the severity of asthma and consists of a series of 7 questions which patients answer on a scale of 0-6, where 0 is no impairment and 6 is maximum impairment. The ACQ score is the mean value of the answers to the 7 questions and patients having a score of <1 are considered to have adequately controlled asthma, whereas patients with a score of ≧1.5 are considered to have poorly controlled asthma.

There appears to be some correlation between subjects who have airway eosinophilia and/or atopic status and subjects who have an ACQ score of ≧1.5. In one embodiment, the presence of airway eosinophilia and/or atopic status is determined by an ACQ score of at least 1.5. In another embodiment, the subject has an ACQ score of at least about 2.0, at least about 2.5, at least about 3.0, at least about 3.5, at least about 4.0, at least about 4.5, at least about 5.0, at least about 5.5, or at least about 6.0.

In one embodiment, the atopic status is determined by immunoglobulin E (IgE) levels. In another embodiment, the atopic status is determined by blood IgE level. In another embodiment, the atopic status is determined by serum IgE level. IgE levels may be measured by any method known to one of skill in the art.

In one embodiment, a subject has a serum IgE level of between about 50 units/mL and 1000 units/mL prior to the administration of the one or more doses of a compound of Formula (I). In another embodiment, a subject has a serum IgE level between 100 and 1000 units/mL, between 150 and 1000 units/mL, between 200 and 1000 units/mL, between 250 and 1000 units/mL, between 300 and 1000 units/mL, between 400 and 1000 units/mL, between 500 and 1000 units/mL, between 50 and 500 units/mL, between 100 and 500 units/mL, between 100 and 400 units/mL, between 150 and 500 units/mL, or between 200 and 500 units/mL prior to the administration of the one or more doses of a compound of Formula (I). In another embodiment, a subject has a serum IgE level of at least 50 units/mL, at least 100 units/mL, at least 150 units/mL, at least 200 units/mL, at least 250 units/mL, at least 300 units/mL, at least 400 units/mL, or at least 500 units/mL prior to the administration of the one or more doses of a compound of Formula (I). In another embodiment, a subject has a serum IgE level of 50 units/mL prior to the administration of a compound of Formula (I). In another embodiment, a subject has a serum IgE level of 100 units/mL prior to the administration of the one or more doses of a compound of Formula (I). In another embodiment, a subject has a serum IgE level of 150 units/mL prior to the administration of the one or more doses of a compound of Formula (I). In another embodiment, a subject has a serum IgE level of 200 units/mL prior to the administration of the one or more doses of a compound of Formula (I). In another embodiment, a subject has a serum IgE level of 250 units/mL prior to the administration of the one or more doses of a compound of Formula (I). In another embodiment, a subject has a serum IgE level of 300 units/mL prior to the administration of the one or more doses of a compound of Formula (I). In another embodiment, a subject has a serum IgE level of 350 units/ml, prior to the administration of the one or more doses of a compound of Formula (I). In another embodiment, a subject has a serum IgE level of 400 units/mL prior to the administration of the one or more doses of a compound of Formula (I).

In another embodiment, a subject has a serum IgE level of 500 units/mL prior to the administration of the one or more doses of a compound of Formula (I).

In another embodiment, the degree of airway eosinophilia or atopic status is determined by the presence of a Th2 high genomic signature in cells measured by elevated expression of genes for periostin (POSTN), chloride channel regulator 1 (CLCA1), and serine (or cysteine) peptidase inhibitor, clade B, member 2 (SERPINB2). The presence of Th2 high genomic signature may be measured by any method known to one of skill in the art.

The degree of airway eosinophilia and/or atopic status can be determined by a single factor or a combination of factors. In one embodiment, the degree of airway eosinophilia and/or atopic status is based on the skin prick test and sputum eosinophil percentage. In another embodiment, the degree of airway eosinophilia and/or atopic status is determined by observing the skin prick test and the baseline blood eosinophil count. In another embodiment, the degree of airway eosinophilia and/or atopic status is determined by observing the baseline IgE level and the baseline sputum eosinophil percentage. In another embodiment, the degree of airway eosinophilia and/or atopic status is determined by observing the baseline blood eosinophil count and the baseline sputum eosinophil percentage. In another embodiment, the degree of airway eosinophilia and/or atopic status is determined by observing the IgE level, the baseline blood eosinophil count, and the baseline sputum eosinophil percentage. In another embodiment, degree of airway eosinophilia and/or atopic status is determined based on the skin prick test and by observing the baseline IgE level, the baseline blood eosinophil count, and the baseline sputum eosinophil percentage.

The present invention also provides a method for treating a disease or condition in a subject comprising: (a) determining the percentage of eosinophils in a sputum sample of said subject; (b) administering a compound of Formula (I) to said subject if the percentage in (a) is greater than or equal to a percentage of eosinophils determined to indicate a high degree of airway eosinophilia.

The present invention also provides a method for treating a disease or condition in a subject comprising: (a) determining the blood eosinophil count in a subject; (b) administering a compound of Formula (I) to said subject if the count in (a) is greater than or equal to a blood eosinophil count determined to indicate a high degree of airway eosinophilia.

Surprisingly, the inventors have also found that the effectiveness of a compound of Formula (I) in treating eosinophilic or atopic conditions varies according to the age of the subject. Thus, in an embodiment of the invention, the subject is less than or equal to 50 years of age. In another embodiment, the subject is less than or equal to 40 years of age. In another embodiment, the subject is less than or equal to 30 years of age.

Age of onset of the atopic or eosinophilic condition has also proved to be a factor, with a compound of Formula (I) being more effective in the treatment of subjects wherein the age of onset of the atopic or eosinophilic condition is less than or equal to 30 years of age. In one embodiment, the age of onset of the atopic or eosinophilic condition is about 0 to 10 years; in a further embodiment, the age of onset is about 11 to 20 years and in still a further embodiment, the age of onset is about 21 to 30 years.

In another embodiment, atopic status may be determined on the basis of the subject's medical history. Therefore, if the subject has a history of atopic disease, that subject may be considered to have high atopic status. In the case of children, particularly children under about 16 years of age, more particularly children under about 12 years of age or under about 6 years of age, and especially children under 2 years of age, the medical history of the subject's immediate family may also be taken into account. Thus, if one or more parents or siblings of the subject has an atopic condition, the subject may be considered to have high atopic status.

The effectiveness of a compound of Formula (I) in treating an eosinophilic or atopic disease or condition may be determined by measuring a baseline score and comparing it with a score determined after a course of treatment with a compound of Formula (I).

Alternatively, a different test may be used to determine the effectiveness of the treatment with the compound of Formula (I). One suitable measure is the FEV₁ score, which can be determined before and after treatment with the compound of Formula (I).

The term “about” is used herein to mean the given number plus or minus 1 to 10%. The CRTH2 antagonist may be a compound of Formula (I):

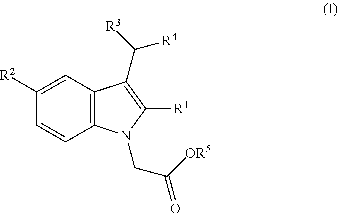

wherein
R¹ is C₁-C₆ alkyl;
R² is halogen;
R³ is aryl or heteroaryl optionally substituted with one or more substituents selected from halo, OH, CN, R⁶, COR⁶, CH₂R⁶, OR⁶, SR⁶, SO₂R⁶ or SO₂YR⁶;
R⁶ is C₁-C₆ alkyl, C₃-C₈ cycloalkyl, heterocyclyl, aryl or heteroaryl, any of which may optionally be substituted with one or more substituents selected from halo, OH, CN, NO₂, C₁-C₆ alkyl or O(C₁-C₆ alkyl);
Y is NH or a straight or branched C₁-C₄ alkylene chain;
R⁴ is H or C₁-C₄ alkyl;
R⁵ is hydrogen, C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂;
m is 1 or 2;
n is 1-4;
X is OR⁷ or N(R⁷)₂;
R⁷ is hydrogen or methyl; and
R⁸ is C₁-C₁₈ alkyl;
or a pharmaceutically acceptable salt, hydrate, solvate, or complex thereof.

The term “C₁-C₆ alkyl” refers to a straight or branched saturated hydrocarbon chain having one to six carbon atoms and optionally substituted with one or more halo substituents or with one or more C₃-C₇ cycloalkyl groups. Examples include methyl, ethyl, n-propyl, isopropyl, t-butyl, n-hexyl, trifluoromethyl, 2-chloroethyl, methylenecyclopropyl, methylenecyclobutyl, methylenecyclobutyl and methylenecyclopentyl. The terms “C₁-C₄ alkyl” and “C₁-C₁₈ alkyl” have similar meanings except that they contain from one to four and from one to eighteen carbon atoms respectively.

The term “C₃-C₇ cycloalkyl” refers to a saturated 3 to 7 membered carbocyclic ring. Examples of such groups include cyclopropyl, cyclobutyl, cyclopentyl and cyclohexyl.

The term “C₁-C₄ alkylene” in the context of the present specification refers to a disubstituted straight or branched saturated hydrocarbon chain having one to four carbon atoms.

The term “halo” refers to fluoro, chloro, bromo or iodo.

The term “aryl” in the context of the present specification refers to an aromatic ring system having from 5 to 14 ring carbon atoms and containing up to three rings. Examples of aryl groups are benzene and naphthalene.

The term “heteroaryl” in the context of the specification refers to a ring system with aromatic character having from 5 to 14 ring atoms, at least one of which is a heteroatom selected from N, O and S, and containing up to three rings. Where a heteroaryl group contains more than one ring, not all rings must be fully aromatic in character. Rings which are not fully aromatic may be substituted with one or more oxo groups. Examples of heteroaryl groups include pyrrole, thiophene, thiazole, pyridine, pyrimidine, indole, benzofuran, benzimidazole, tetrahydroquinoline, indoline, quinoline, isoquinoline, quinoxaline, imidazo[1,2-a]pyridine, pyrazolo[1,5-a]pyridine, 2,3-dihydro-1-benzothiopyrane and 2,3-dihydro-1λ⁶-benzothiopyran-1,1-dione.

The term “heterocyclyl” in the context of the specification refers to a saturated ring system having from 4 to 8 ring atoms, at least one of which is a heteroatom selected from N, O and S and which may be optionally substituted by one or more oxo groups. Examples of heterocyclyl groups include azetidinyl, piperidinyl; tetrahydrofuranyl, tetrahydropyranyl, dioxanyl, thiomorpholinyl, 1,1-dioxo-1λ₆-thiomorpholinyl, morpholinyl, pyrrolyl, piperizinyl, azepanyl, 1,4-diazepanyl, 1,4-oxazepanyl and azocanyl.

Appropriate pharmaceutically and veterinarily acceptable salts of the compounds of Formula (I) include basic addition salts such as sodium, potassium, calcium, aluminium, zinc, magnesium and other metal salts as well as choline, diethanolamine, ethanolamine, ethyl diamine, megulmine and other well known basic addition salts as summarised in J. Med. Chem., 50, 6665-6672 (2007) and/or known to those skilled in the art.

Where appropriate, pharmaceutically or veterinarily acceptable salts may also include salts of organic acids, especially carboxylic acids, including but not limited to acetate, trifluoroacetate, lactate, gluconate, citrate, tartrate, maleate, malate, pantothenate, adipate, alginate, aspartatc, benzoate, butyrate, digluconate, cyclopentanate, glucoheptanate, glycerophosphate, oxalate, heptanoate, hexanoate, fumarate, nicotinate, pamoate, pectinate, 3-phenylpropionate, picrate, pivalate, proprionate, tartrate, lactobionate, pivolate, camphorate, undecanoate and succinate, organic sulfonic acids such as methanesulfonate, ethanesulfonate, 2-hydroxyethane sulfonate, camphorsulfonate, 2-naphthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids.

Salts which are not pharmaceutically or veterinarily acceptable may still be valuable as intermediates.

In the present invention, references to an element are intended to cover all isotopes of that element. For example the terms “H” and “hydrogen” encompass not only ¹H but also deuterium and tritium. Similarly references to “C” and carbon encompass all isotopes, for example ¹²C and ¹⁴C.

The CRTH2 antagonists of Formula (I) are indole-1-acetic acid derivatives and are some of the compounds and analogues thereof described in WO2005/044260, WO2005/094816, WO2006/095183, WO2008/012511, PCT/GB2009/000142, PCT/GB2009/000171, PCT/GB2009/000175 and UK patent application No 0801674.3, filed 30 Jan. 2008. The indole-1-acetic acid derivatives of Formula (I) as defined above and methods for their preparation are described in detail in these documents.

In one embodiment of the invention, the compound of Formula (I) is a CRTH2 antagonist in which R⁵ is hydrogen.

In an alternative embodiment of the invention, the compound of Formula (I) is a prodrug for a CRTH2 antagonist and R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂; where
m is 1 or 2;
n is 1-4;
X is R⁷ or N(R⁷)₂;
R⁷ is hydrogen or methyl; and
R⁸ is C₁-C₁₈ alkyl.

In suitable compounds of Formula (I), independently or in any combination:

R¹ is fluoro;
R² is C₁-C₄ alkyl, particularly methyl or ethyl but more especially methyl;
R⁴ is H or methyl; and
R³ is quinoline, quinoxaline, isoquinoline, thiazole, phenyl, naphthalene, thiophene, pyrrole or pyridine, any of which may optionally be substituted as set out above.

In particularly suitable compounds, R⁴ is H.

More typical R³ groups include optionally substituted quinoline, phenyl, naphthalene, thiophene, pyrrole or pyridine.

When R³ is quinoline or isoquinoline, it is suitably unsubstituted or substituted with one or more halo substituents, especially fluoro.

When R³ is phenyl, naphthalene, thiophene, pyrrole or pyridine, it may optionally have one or more substituents, with particularly suitable substituents including OR⁶, SO₂R⁶ or SO₂YR⁶; where R⁶ and Y are as defined above.

Typically, in this case, R⁶ is C₁-C₆ alkyl, a 4- to 6-membered cycloalkyl group, a 5- or 6-membered heterocyclyl group or phenyl, any of which may be substituted as defined above.

When R³ is pyridyl it is most suitably a 3-pyridyl moiety.

In more active compounds, Y, when present, is a CH₂ moiety.

When R³ is substituted with SO₂R⁶ or SO₂YR⁶, the R⁶ group is generally unsubstituted or substituted with one or more substituents chosen from methyl and halo, particularly chloro or fluoro.

When R³ is substituted with OR⁶, the R⁶ group may be unsubstituted or substituted with one or more substituents chosen from halo, cyano, C₁-C₄ alkyl and O(C₁-C₄ alkyl).

Particularly suitable compounds of Formula (I) include:
{3-[1-(4-Chloro-phenyl)-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
{5-Fluoro-2-methyl-3-(1-naphthalen-2-yl-ethyl)-indol-1-yl}-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl)-acetic acid;
(3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methylbenzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-{[2-(Benzenesulfonyl)phenyl]methyl}-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
(5-Fluoro-3-[(1-methanesulfonylnaphthalen-2-yl)methyl]-2-methylindol-1-yl)-acetic acid;
{5-Fluoro-3-[(6-methanesulfonylnaphthalen-2-yl}methyl]-2-methylindol-1-yl)-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl)-acetic acid;
(5-Fluoro-2-methyl-3-(pyrazolo[1,5-a]pyridin-3-ylmethyl}indol-1-yl)-acetic acid;
(5-Fluoro-3-(imidazo[1,2-a]pyridin-2-ylmethyl)-2-methylindol-1-yl)-acetic acid;
(5-Fluoro-2-methyl-3-([2-(methylsulfanyl)phenyl]methyl)indol-1-yl)-acetic acid;
(5-Fluoro-2-methyl-3-{[3-(methylsulfanyl)phenyl]methyl}indol-1-yl)-acetic acid;
(5-Fluoro-2-methyl-3-([4-(ethylsulfanyl)phenyl]methyl)indol-1-yl)-acetic acid
(3-{[4-(Ethylsulfanyl)phenyl]methyl}-5-fluoro-2-methylindol-1-yl)-acetic acid;
(5-Fluoro-2-methyl-3-([4-(n-propylsulfanyl)phenyl]methyl)indol-1-yl)-acetic acid;
(5-Fluoro-2-methyl-3-([4-(i-propylsulfanyl)phenyl]methyl)indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-([4-(pentan-3-ylsulfanyl)phenyl]methyl)indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-{[2-(pentane-1-sulfonyl)phenyl]methyl}indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-([3-(Butylsulfamoyl)phenyl]methyl-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl)indol-1-yl)-acetic acid;
(5-Fluoro-2-methyl-3-[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-([4-(propylsulfamoyl)phenyl]methyl)indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
(5-Fluoro-2-methyl-3-[(3-phenoxyphenyl)methyl]indol-1-yl)-acetic acid;
[5-Fluoro-3-({4-[(4-fluorophenyl)carbonyl]-1-methylpyrrol-2-yl}methyl)-2-methylindol-1-yl]-acetic acid;
{5-Fluoro-2-methyl-3-[(6-{[3-(trifluoromethyl)phenyl]methyl}pyridin-3-yl)methyl]indol-1-yl}-acetic acid;
(5-Fluoro-2-methyl-3-[(3-phenoxythiophen-2-yl)methyl]indol-1-yl)-acetic acid;
(3-([2-(Benzenesulfonyl)-1,3-thiazol-5-yl]methyl)-5-fluoro-2-methylindol-1-yl)-acetic acid;
(3-[(1-Benzylpyrazol-4-yl)methyl]-5-fluoro-2-methylindol-1-yl)-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
   or the C₁-C₆ alkyl, aryl, (CH₂)ₘOC(═O)C₁-C₆alkyl, ((CH₂)ₘO)ₙCH₂CH₂X, (CH₂)ₘN(R⁷)₂ or CH((CH₂)ₘO(C═O)R⁸)₂ esters of any of the above; wherein
      m is 1 or 2;
      n is 1-4;
      X is OR⁷ or N(R⁷)₂;
      R⁷ is hydrogen or methyl;
      R⁸ is C₁-C₁₈ alkyl.

The compounds of Formula (I) may be prepared according to methods set out in the prior art or methods analogous to those set out in the prior art, in particular WO2005/044260, WO2005/094816, WO2006/095183, WO2008/012511, PCT/GB2009/000142, PCT/GB2009/000171, PCT/GB2009/000175 and UK patent application No. 0801674.3.

The compounds of Formula (I) are useful in the treatment and prevention of eosinophilic diseases and conditions including eosinophilic asthma, atopic asthma, uncontrolled asthma, eosinophilic chronic obstructive pulmonary disease (COPD), eosinophilic nasal polyps, eosinophilic oesophagitis, eosinophilic atopic dermatitis, eosinophilic allergic conjunctivitis, allergic rhinitis, and Churg Strauss sydrome. In one embodiment, the disease is eosinophilic asthma. In another embodiment, the disease is atopic asthma. In another embodiment, the disease is uncontrolled asthma. In another embodiment, the disease is chronic obstructive pulmonary disease. In another embodiment, the disease is allergic rhinitis.

The CRTH2 antagonist of Formula (I) will generally be administered in a pharmaceutical formulation. The type of formulation chosen will depend upon the atopic condition suffered by the patient and the type of infection to be treated or prevented. Examples of formulations include those suitable for oral, rectal, nasal, bronchial (inhaled), topical (including eye drops, buccal and sublingual), vaginal or parenteral (including subcutaneous, intramuscular, intravenous and intradermal) administration. The formulations may be prepared by any methods well known in the art of pharmacy.

As mentioned above, the route of administration will depend upon the condition to be treated but preferred compositions are formulated for oral, nasal, bronchial or topical administration.

The formulation may be prepared by bringing into association the above defined active agent with a carrier. In general, the formulations are prepared by uniformly and intimately bringing into association the active agent with liquid carriers or finely divided solid carriers or both, and then if necessary shaping the product.

Formulations for oral administration in the present invention may be presented as: discrete units such as capsules, sachets, tablets, which may be chewable tablets, or lozenges, each containing a predetermined amount of the active agent; as a powder or granules; as fine particles for sprinkling over food; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets, capsules, formulations comprising a mucoadherent, etc.), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Some formulations may comprise a mucoadherent, for example a mucopolysaccharide such as sodium hyaluronate. Such compositions may be a formulation as, for example, liquids, liquid syrups, soft gels, liquid gels, flowable gels, or aqueous suspensions and may, in addition to the active agent and the mucoadherent, also contain one or more additional excipients as set out above. Liquid formulations will usually also contain a liquid carrier, which may be a solvent or suspending agent, for example, water or saline solution, and may also contain a substance to increase their viscosity, for example, sodium carboxymethylcellulose, sorbitol, or dextran.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, CRTH2 antagonists such as compounds of Formula (I) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

For topical application to the eye, CRTH2 antagonists such as compounds of Formula (I) may be made up into an eye drop formulation. Suitable eye drop formulations are also well known in the art and are described in standard text books of pharmaceutics such as the British Pharmacopoeia.

CRTH2 antagonists which are intended to treat or prevent severe exacerbations of asthma or to treat or prevent respiratory viral infections in asthma and COPD patients may be administered to the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the oral dose of the compound will be about 0.01 to 100 mg/kg; so as to achieve a concentration of drug in the plasma at a concentration effective to inhibit PGD₂ at the CRTH2 receptor. The precise amount of a compound of CRTH2 antagonist which is therapeutically effective, and the route by which such compound is best administered, is readily determined by one of ordinary skill in the art by comparing the blood level of the agent to the concentration required to have a therapeutic effect.

In formulations for topical administration, such as eye drops, creams or ointments, the concentration of active compound will typically be about 0.1%-1% w/w such that systemic exposure of the CRTH2 antagonist compound is limited.

Compounds of Formula (I) may be used in combination with one or more active agents which are useful in the treatment of infection and such agents may therefore be included in the pharmaceutical composition. There is therefore provided a pharmaceutical composition comprising a compound of Formula (I) in combination with one or more agents which are useful in the treatment of infection.

Examples of agents useful in the treatment of infections include anti-viral agents, interferons, anti-RSV antibodies and antibiotics.

Therefore, there is also provided a product comprising a compound of Formula (I) and one or more of an anti-viral agent, an interferon or an anti-RSV antibody as a combined preparation for simultaneous, separate or sequential use in the treatment of infection in a patient suffering from an atopic condition.

Since the mechanism of action appears to be that described above, the treatment or prevention of infection will observed with any CRTH2 antagonist. Documents which discuss the use of CRTH2 antagonists for the treatment of Th2-dependent allergic diseases include WO-A-03/066046, WO-A-03/066047, WO-A-03/097042, WO-A-03/097598, WO-A-03/101981, WO-A-03/101961, WO-A-2004/007451, WO-A-2005/019171, WO-A-2005/054232, WO-A-2004/089884, WO-A-2004/089885, WO-A-2005/018529, WO-A-2006/005909, WO2006/021759, WO-A-2007/039736, WO-A-2007/052023, WO-A-2006/075139, WO-A-2007/068894, WO-A-2007138282, WO-A-2008/119917, WO-A-2008/113965, WO-A-2008/074966, WO-A-2008/078069, WO-A-2007/144625, WO-A-2007/028999, WO-A-2007/031747, WO-A-2006/136859, WO-A-2006/111560, WO-A-2005/094816, WO-A-2005/040112, WO-A-2005/040114, WO-A-2004/096777, WO-A-2005/123731, WO-A-2006/125784, WO-A-2007/045867, WO-A-2006/034419, WO-A-2006/036994, WO-A-2007/022501, WO-A-2004/106302, WO-A-2004/032848, WO-A-2005/100321, WO-A-2006/091674, WO-A-2004/0.58164, WO-A-2005/007094, WO-A-2007/036743, WO-2004/035543, WO-A-2007/062797, WO-A-2007/062773, WO-A-2007/062678, WO-A-2007/062677, WO-A-2005/116001, WO-A-2005/115382, WO-A-2005/115374, WO-A-2006/111560, WO-A-2006/037982, WO-A-2006/056752, WO-A-2007/039741, WO-A-2005/073234, WO-A-2005/105727, WO-A-2006/063763, WO-A-2006/125593 and WO-A-2006/125596.

Some particularly suitable compounds are compounds which are similar to the compounds of Formula (I) but in which the moiety —CHR³R⁴ of Formula (I) is replaced by R³, SR³, SOR³ or SO₂R³, where R³ is as defined for Formula (I).

In one embodiment, the compound of Formula (I) is administered in combination with one or more additional agent which is of use in the treatment of allergic or inflammatory diseases.

In one embodiment, the additional agent administered in combination with the compound of Formula (I) is selected from the group consisting of montelukast; (3 adrenoreceptor agonists such as metaproterenol, isoproterenol, isoprenaline, albuterol, salbutamol, formoterol, salmeterol, terbutaline, orciprenaline, bitolterol mesylate, pirbuterol, and indacaterol; inhaled corticosteroids such as prednisone, prednisolone, flunisolide, triamcinolone acetonide, beclomethasone dipropionate, budesonide, fluticasone propionate, fluticasone furoate, mometasone furoate, and ciclesonide; muscarinic receptor antagonists such as ipratropium and tiotropium; anti-IL-5 antibodies such as mepolizumab and reslizuniab; anti-IgE antibodies such as omalizumab; and agents which block the actions of IL-4 and/or IL-13 such as the IL-4 mutein pitrakinra and anti-IL-13 receptor α antibodies such as AMG-317 and anti-IL-13 antibodies.

In the context of the present invention, a CRTH2 antagonist is a compound which binds to the CRTH2 receptor with a Ki of less than 1000 nM in the radioligand binding assay described below and which inhibits the dose-dependent increase in intracellular Ca²⁺ mobilisation in CHO/CRTH2 cells treated with PGD₂ with an IC₅₀ of less than 1000 nM. Although any compound with CRTH2 antagonist activity would be suitable for use in treating a subject to allergens, the CRTH2 antagonist is suitably a compound of Formula (I).

### EXAMPLES

Having now generally described this invention, the same will be understood by reference to the following examples which are provided herein for purposes of illustration only and are not intended to be limiting unless otherwise specified.

### Example 1

### 4 Week Study in Patients with Mild to Moderate Asthma

### Study Design

The study was a randomized, double blind, placebo controlled study of (5-fluoro-2-methyl-3-quinolin-2-ylmethyl-indol-1-yl)-acetic acid (OC000459) for 28 days in patients with mild to moderate asthma with a FEV₁ of 60-80% of predicted and requiring only short acting inhaled β₂ agonists for symptomatic control. The study compared patients taking 100 mg of OC000459 twice daily with patients taking a placebo twice daily. The study consisted of 112 patients with 53 patients taking OC000459 and 59 patients taking the placebo.

### Study Population

The following selection criteria were used to identify subjects:

### Inclusion Criteria:

1. Males and females aged 18-55 years.
2. Asthma controlled by short-acting β₂ agonists only.
3. Mild to moderate persistent asthma according to GINA4 guidelines for at least 12 months.
4. Non-smokers for at least the past 12 months with a pack history of less than 10 pack years.
5. History of asthma symptoms increasing in response to external allergens.
6. Testing positive on skin prick test to an allergen associated with the patient's allergen.

### Exclusion Criteria:

1. Receipt of prescribed or over-the-counter medication within 14 days prior to the first study day.
2. Use of inhaled or local corticosteroids in the period 28 days prior to screening.

### Results

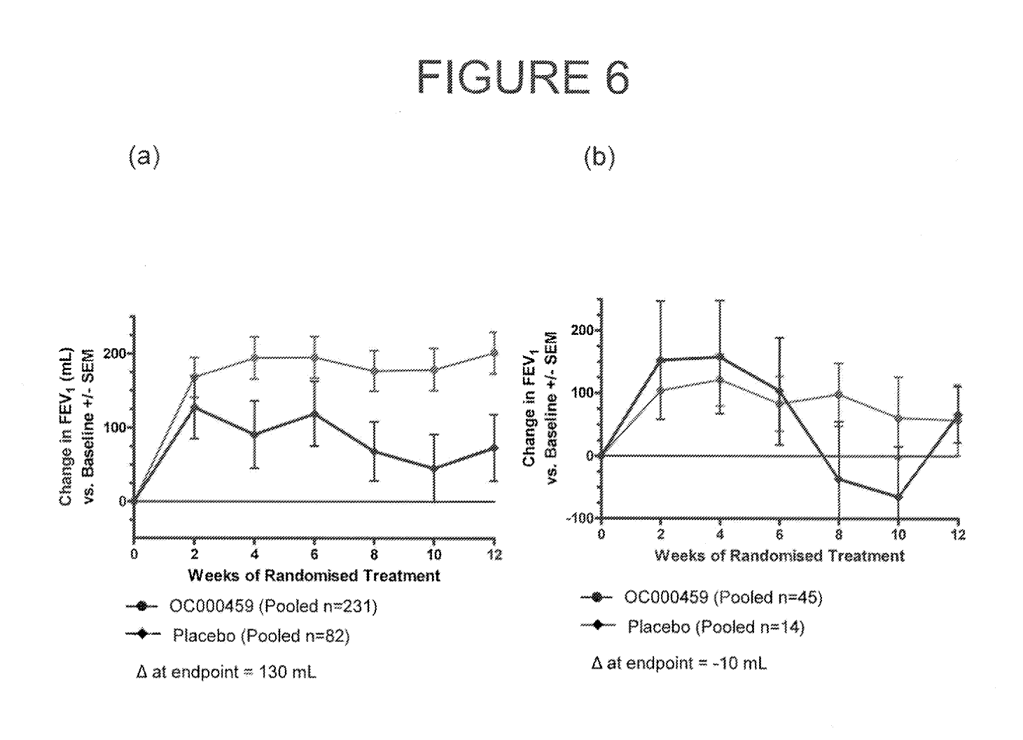

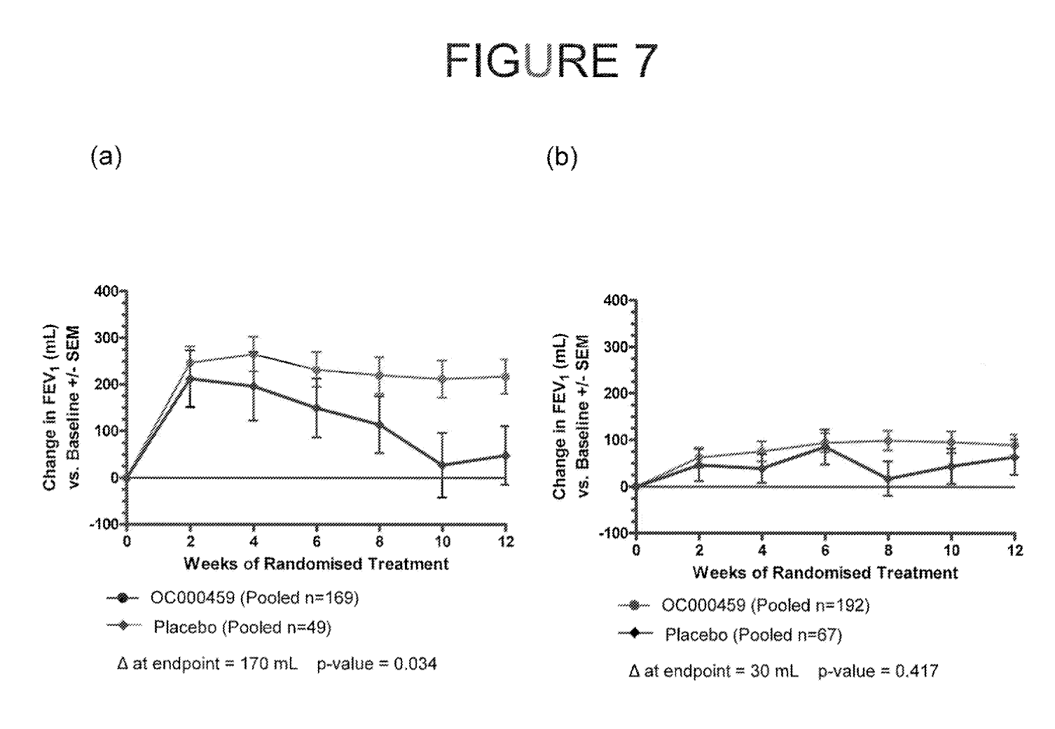

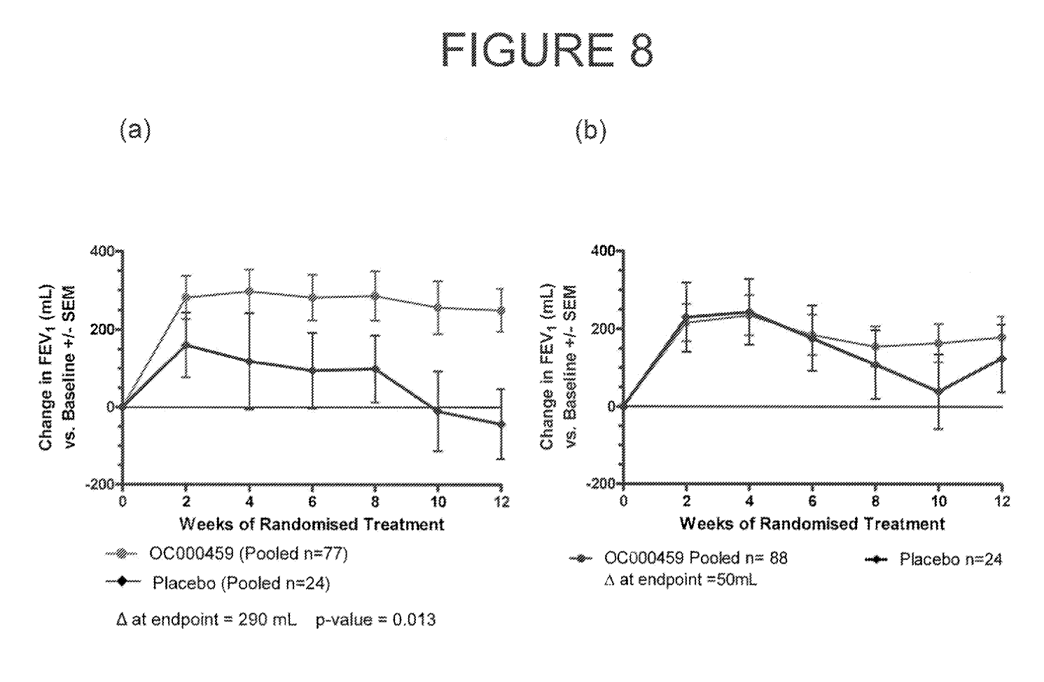

FIG. 6 shows the results of the 112 patients (53 patients given OC000459 and 59 patients given placebo). As shown in FIG. 6(a), after 4 weeks patients given OC000459 showed a 190 mL improvement in FEV₁ over baseline and a 100 mL improvement in FEV₁ over patients given the placebo. FIG. 6(b) shows the results of 72 patients (34 patients given OC000459 and 38 patients given the placebo) with a blood eosinophil count of greater than or equal to 250/μL. After 4 weeks, patients given OC000459 showed a 250 mL improvement in FEV₁ over baseline and a 180 mL improvement in FEV₁ over patients given the placebo. FIG. 6(c) shows the results of 40 patients (19 patients given OC000459 and 21 patients given the placebo) with a blood eosinophil count of less than 250/μL. After 4 weeks, patients given OC000459 showed only a small improvement over patients given the placebo. Therefore, comparing the results in FIG. 6(b) and FIG. 6(c), patients with a higher blood eosinophil count showed a greater improvement in FEV₁.

### Example 2

### 12 Week Study in Patients with Mild to Moderate Persistent Asthma

### Study Design

The study was a randomized, double blind, placebo controlled, four arm study of three dosage levels of (5-fluoro-2-methyl-3-quinolin-2-ylmethyl-indol-1-yl)-acetic acid (OC000459) tablets in patients with asthma controlled by β₂ agonists alone. The study compared patients on OC000459 at three different dose levels (25 mg once daily, 100 mg twice daily, and 200 mg once daily) with patients on placebo after dosing for 12 weeks. The study consisted of 460 patients which yielded 440 evaluable results with 110 patients per arm. There was a screening period of 1 to 2 weeks, followed by placebo run-in for 3 weeks before the treatment period. There was a placebo wash-out for two weeks following the treatment period with a follow-up 3 to 5 weeks after the treatment period.

### Study Population

The following selection criteria were used to identify subjects:

### Inclusion Criteria:

1. Males and females aged 18-55 years.
2. Asthma controlled by short-acting β₂ agonists only.
3. Mild to moderate persistent asthma according to GINA4 guidelines for at least 12 months.
4. Non-smokers for at least the past 12 months with a pack history of less than 10 pack years.

### Exclusion Criteria:

Receipt of prescribed or over-the-counter medication within 14 days prior to the first study day.

### Randomization Criteria:

1. Morning FEV₁ of 60-85% of predicted.
2. Reversibility of ≧12% after salbutamol.
3. Greater than 1 puff per day of salbutamol required.

### Results

Of the 360 patient receiving OC000459, 72% were determined to have a baseline FEV₁ measurement of 60-80% and 32% were determined to have a baseline blood eosinophil count of greater than 250/μL as shown in Table 1. As shown in Table 1, patients with a baseline blood eosinophil count of greater than 250/μL showed a greater improvement in FEV₁ than patients with a lower blood eosinophil count.

TABLE 1
Patient population on OC000459.

| | | 60-80% FEV₁ | Hi-eosinophil count |
|---|---|---|---|
| | All-Comers | Atopic | (>250/μL) |
| % Patients in Study | 100% | 72% | 32% |
| FEV₁ Improvement | 100 mL | 130 mL | 188 mL |

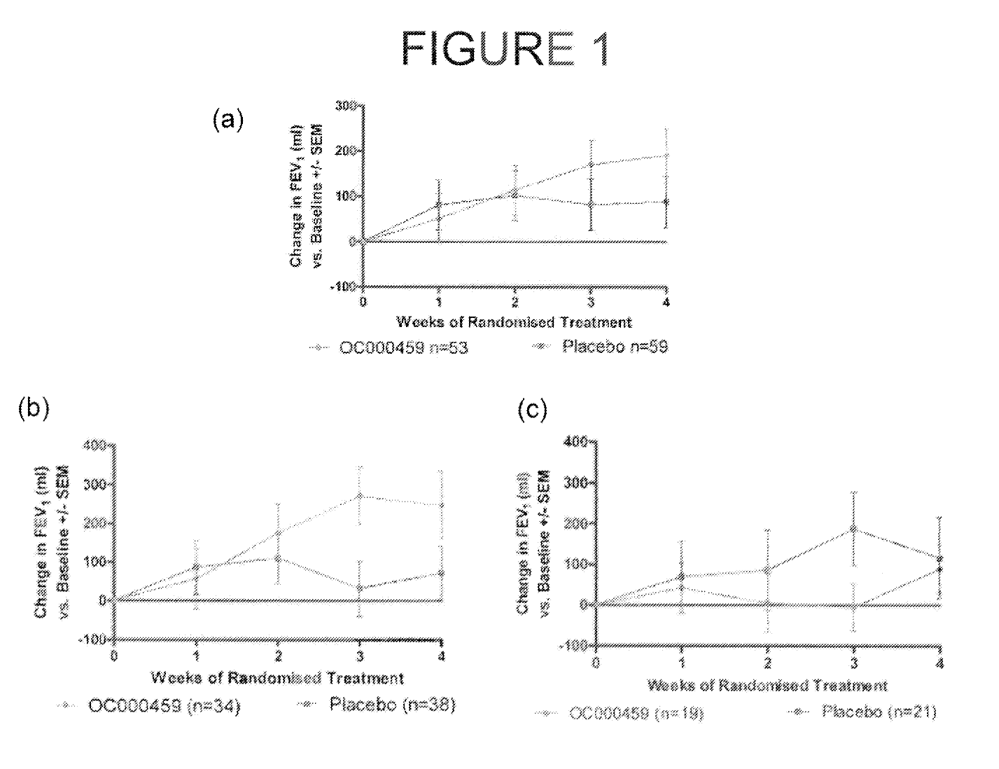

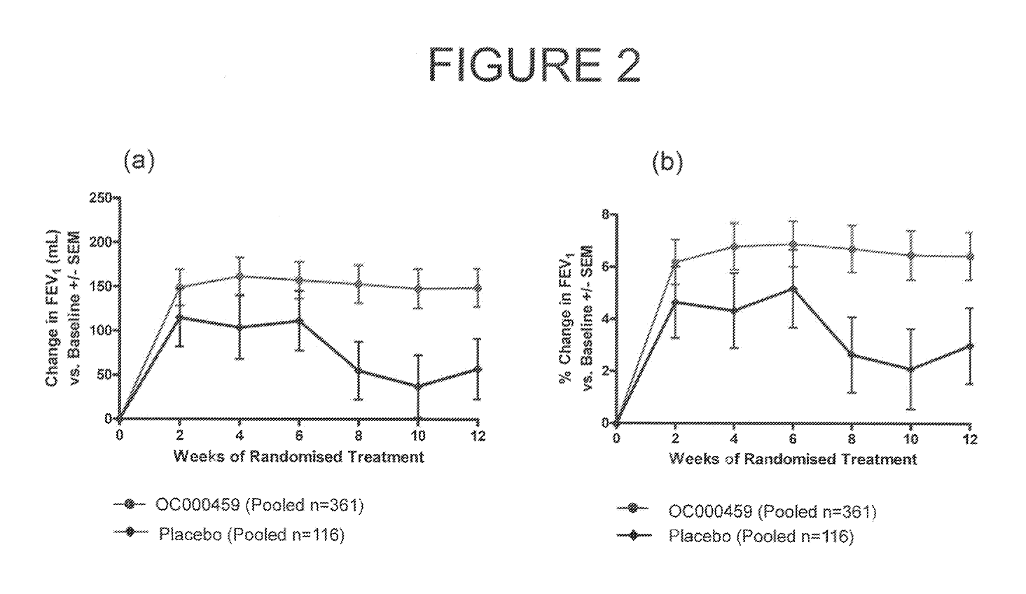

FIG. 1 shows the results of 353 patients (260 patients given OC000459 and 93 patients given placebo) with a FEV₁ measurement of 60-80% of predicted and a positive skin prick test at baseline. After 12 weeks, patients given OC000459 showed a 191 mL improvement in FEV₁ over baseline and a 130 mL improvement in FEV₁ over patients given the placebo. FIG. 2 shows the results of 163 patients (119 patients given OC000459 and 44 patients given the placebo) with a FEV₁ measurement of 60-80% of predicted, a blood eosinophil count of greater than or equal to 250/μL, and a positive skin prick test at baseline. After 12 weeks, patients given OC000459 showed a 184 mL improvement in FEV₁ over baseline and a 188 mL improvement in FEV₁ over patients given the placebo. Therefore, patients with a higher blood eosinophil count showed a greater improvement.

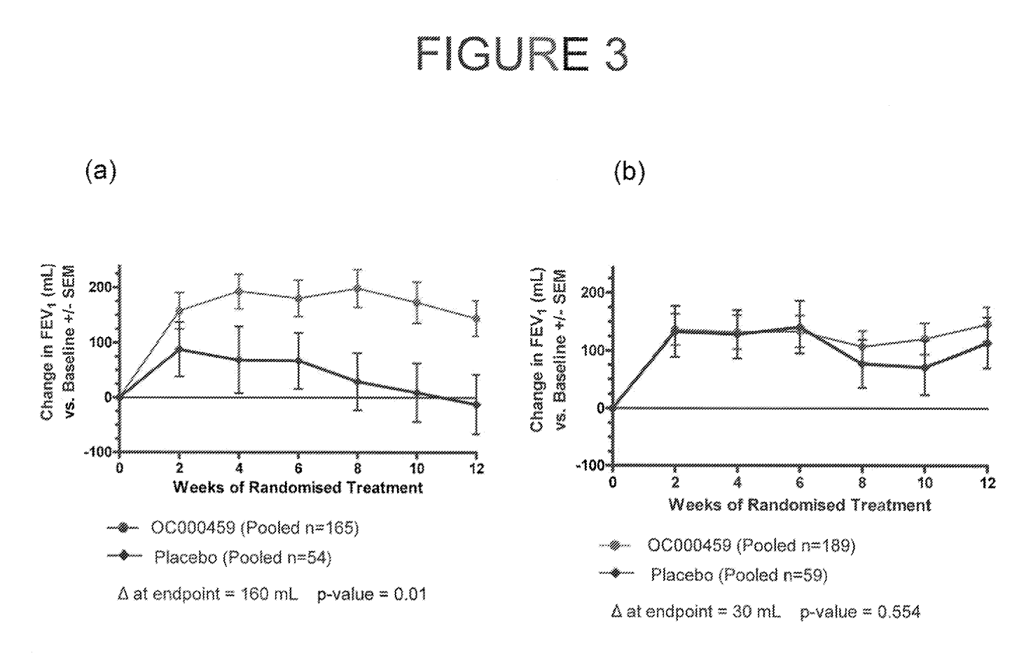

FIG. 3 shows the results of 183 patients (137 patients given OC000459 and 46 patients given the placebo) with a FEV₁ measurement of 60-80% of predicted, a blood eosinophil count of less than 250/μL, and a positive skin prick test at baseline. After 12 weeks, patients given OC000459 showed a 76 ml, improvement in FEV₁ over patients given the placebo. Therefore, comparing the results in FIG. 2 and FIG. 3, patients with a higher blood eosinophil count showed a greater improvement in FEV₁.

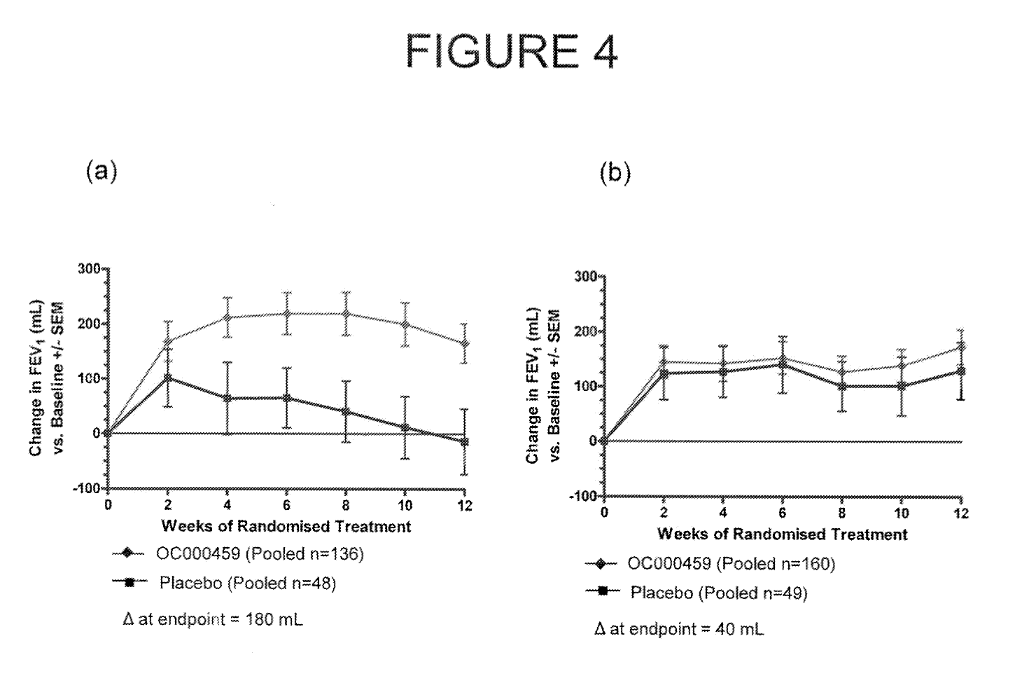

FIG. 4 shows the results of 308 patients (228 patients given OC000459 and 80 patients given the placebo) with an ACQ (asthma control questionnaire) score greater than or equal to 1.5 at baseline. After 12 weeks, patients given OC000459 showed a 140 mL improvement in ACQ score over patients given the placebo.

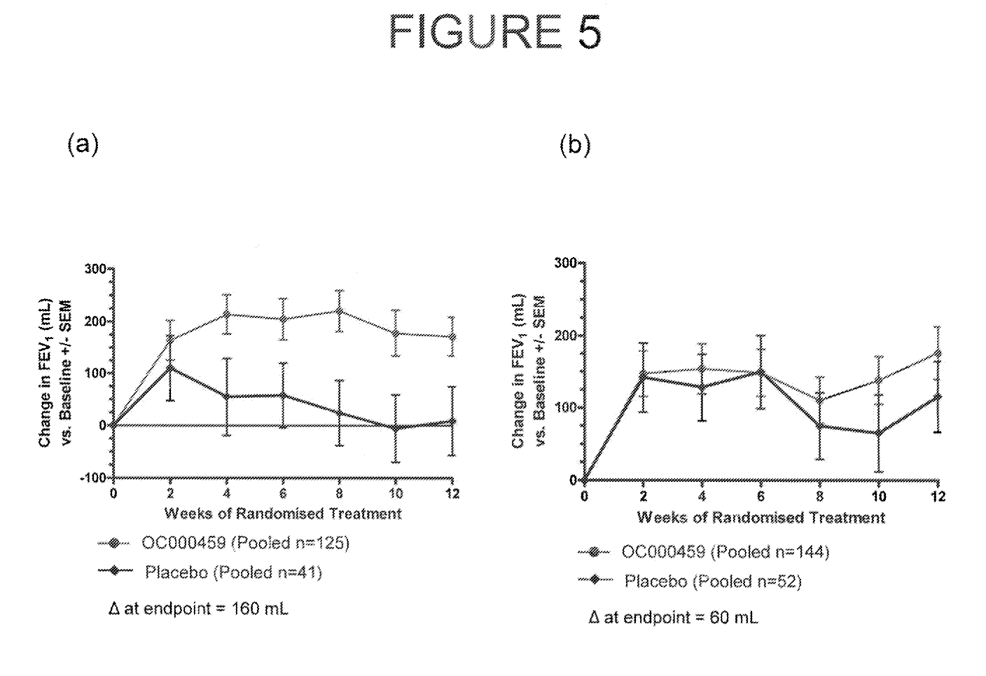

FIG. 5 shows the results of 88 patients (69 patients given OC000459 and 19 patients given the placebo) with an ACQ (asthma control questionnaire) score less than 1.5 at baseline. After 12 weeks, patients given OC000459 showed a 30 mL improvement in ACQ score over patients given the placebo. Therefore, comparing the results in FIG. 4 and FIG. 5, patients with a higher ACQ score showed a greater improvement in FEV₁.

Having now fully described this invention, it will be understood by those of ordinary skill in the art that the same can be performed within a wide and equivalent range of conditions, formulations and other parameters without affecting the scope of the invention or any embodiment thereof. All patents, patent applications, and publications cited herein are fully incorporated by reference herein in their entirety.

1. A method of treating an eosinophilic disease or condition in a subject with a high degree of airway eosinophilia, comprising:
(a) determining the degree of airway eosinophilia in the subject;
(b) administering a compound of Formula (I) to said subject if the degree of airway eosinophilia in (a) is at or above a level determined to be high;
wherein the compound of Formula (I): 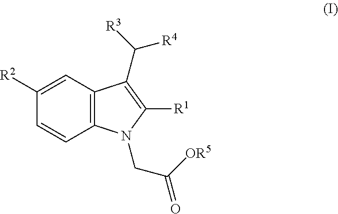
wherein
R¹ is C₁-C₆ alkyl;
R² is halogen; and
R³ is aryl or heteroaryl optionally substituted with one or more substituents selected from halo, OH, CN, R⁶, COR⁶, CH₂R⁶, OR⁶, SR⁶, SO₂R⁶ or SO₂YR⁶;
R⁶ is C₁-C₆ alkyl, C₃-C₈ cycloalkyl, heterocyclyl, aryl or heteroaryl, any of which may optionally be substituted with one or more substituents selected from halo, OH, CN, NO₂, C₁-C₆ alkyl or O(C₁-C₆ alkyl); and
Y is NH or a straight or branched C₁-C₄ alkylene chain;
R⁴ is H or C₁-C₄ alkyl;
R⁵ is hydrogen, C₁-C₆ alkyl, aryl, (CH₂)ₘOC(═O)C₁-C₆alkyl, ((CH₂)ₘO)ₙCH₂CH₂X, (CH—₂)ₘN(R⁷)₂ or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
or a pharmaceutically acceptable salt, hydrate, solvate, or complex thereof.

2. The method of claim 1, wherein R⁵ is hydrogen.

3. The method of claim 1, wherein R⁵ is C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂.

4. The method of claim 1, wherein:
R¹ is C₁-C₄ alkyl;
R² is fluoro;
R³ is optionally substituted and is quinoline, quinoxaline, isoquinoline, thiazole, phenyl, naphthalene, thiophene, pyrrole, or pyridine; and
R⁴ is H or methyl.

5. The method of claim 4, wherein R⁴ is H.

6. The method of claim 1, wherein R³ is optionally substituted and is quinoline, isoquinoline, phenyl, naphthalene, thiophene, pyrrole, or pyridine.

7. The method of claim 6, wherein R³ is quinoline or isoquinoline, wherein the quinoline or isoquinoline is unsubstituted or substituted with one or more halo substituents.

8. The method of claim 6, wherein R³ is optionally substituted with one or more substituents and is phenyl, naphthalene, thiophene, pyrrole, or pyridine, wherein the one or more substituents are OR⁶, SO₂R⁶ or SO₂YR⁶.

9. The method of claim 1, wherein R⁶ is optionally substituted and is C₁-C₆ alkyl, a 4- to 6-membered cycloalkyl group, a 5- or 6-membered heterocyclyl group, or phenyl.

10. The method of claim 6, wherein R³ is a 3-pyridyl moiety.

11. The method of claim 1, wherein R³ is substituted with SO₂YR⁶, wherein Y is a CH₂ moiety.

12. The method of claim 1, wherein R³ is substituted with SO₂R⁶ or SO₂YR⁶, wherein the R⁶ group is unsubstituted or substituted with one or more substituents selected from methyl and halo.

13. The method of claim 1, wherein R³ is substituted with OR⁶, wherein the R⁶ group is unsubstituted or substituted with one or more substituents selected from the group consisting of halo, cyano, C₁-C₄ alkyl, and O(C₁-C₄ alkyl).

14. The method of claim 1, wherein the compound of Formula (I) is:
{3-[1-(4-Chloro-phenyl-ethyl]-5-fluoro-2-methyl-indol-1-yl}-acetic acid;
{5-Fluoro-2-methyl-3-[1-(4-trifluoromethyl-phenyl)-ethyl]-indol-1-yl}-acetic acid;
{3-[1-(4-tert-Butyl-phenyl)-ethyl]-5-fluoro-2-methyl-indol-1-yl}-acetic acid;
{5-Fluoro-3-[1-(4-methanesulfonyl-phenyl)-ethyl]-2-methyl-indol-1-yl}-acetic acid;
[5-Fluoro-2-methyl-3-(1-naphthalen-2-yl-ethyl)-indol-1-yl]-acetic acid;
(5-Fluoro-2-methyl-3-quinolin-2-ylmethyl-indol-1-yl)-acetic acid;
(5-Fluoro-2-methyl-3-naphthalen-2-ylmethyl-indol-1-yl)-acetic acid;
[5-Fluoro-3-(8-hydroxyquinolin-2-ylmethyl)-2-methyl-indol-1-yl]-acetic acid;
[5-Fluoro-2-methyl-3-(quinoxalin-2-ylmethyl)indol-1-yl]-acetic acid;
[5-Fluoro-3-(4-methoxy-benzyl)-2-methyl-indol-1-yl]-acetic acid;
[5-Fluoro-2-methyl-3-(1,3-thiazol-2-ylmethyl)indol-1-yl]-acetic acid;
[3-(4-Chloro-benzyl)-5-fluoro-2-methyl-indol-1-yl]-acetic acid;
[5-Fluoro-2-methyl-3-(4-trifluoromethyl-benzyl)-indol-1-yl]-acetic acid;
[5-Fluoro-2-methyl-3-(4-tert-butyl-benzyl)-indol-1-yl]-acetic acid;
{5-Fluoro-2-methyl-3-[(4-phenylphenyl)methyl]indol-1-yl}-acetic acid;
[5-Fluoro-3-(4-methanesulfonyl-benzyl)-2-methyl-indol-1-yl]-acetic acid;
{5-Fluoro-3-[(6-fluoroquinolin-2-yl)methyl]-2-methylindol-1-yl}-acetic acid;
(2-Methyl-3-quinolin-2-ylmethyl-indol-1-yl)-acetic acid;
(5-Chloro-2-methyl-3-quinolin-2-ylmethyl-indol-1-yl)-acetic acid;
(3-{[1-(Benzenesulfonyl)pyrrol-2-yl]methyl}-5-fluoro-2-methylindol-1-yl)-acetic acid;
[5-Fluoro-2-methyl-3-({1-[(4-methyl benzene)sulfonyl]pyrrol-2-yl}methyl)indol-1-yl]-acetic acid;
[3-({1-[(2,4-Difluorobenzene)sulfonyl]pyrrol-2-yl}methyl)-5-fluoro-2-methylindol-1-yl]-acetic acid;
(3-([2-(Benzenesulfonyl)phenyl]methyl)-5-fluoro-2-methylindol-1-yl)-acetic acid;
[3-({2-[(4-Chlorobenzene)sulfonyl]phenyl}methyl)-5-fluoro-2-methylindol-1-yl]-acetic acid;
[5-Fluoro-3-({2-[(4-fluorobenzene)sulfonyl]phenyl}methyl)-2-methylindol-1-yl]-acetic acid;
(3-{[2-(Benzenesulfonyl)pyridin-3-yl]methyl}-5-fluoro-2-methylindol-1-yl)-acetic acid;
[5-Fluoro-3-({2-[(4-fluorobenzene)sulfonyl]pyridin-3-yl}methyl)-2-methylindol-1-yl]-acetic acid;
[3-({2-[(4-Chlorobenzene)sulfonyl]pyridin-3-yl}methyl)-5-fluoro-2-methylindol-1-yl]-acetic acid;
2-(3-(4-(Benzylsulfonyl)benzyl)-5-fluoro-2-methyl-indol-1-yl)-acetic acid;
2-(3-(4-(4-Chlorobenzylsulfonyl)benzyl)-5-fluoro-2-methyl-indol-1-yl)-acetic acid;
2-(3-(3-(Benzylsulfonyl)benzyl)-5-fluoro-2-methyl-indol-1-yl)-acetic acid;
2-(5-Fluoro-3-(3-(4-fluorobenzylsulfonyl)benzyl)-2-methyl-indol-1-yl)-acetic acid;
2-(3-(2-(Benzylsulfonyl)benzyl)-5-fluoro-2-methyl-indol-1-yl)-acetic acid;
2-(3-(4-(4-Fluorobenzylsulfonyl)benzyl)-5-fluoro-2-methyl-indol-1-yl)-acetic acid;
2-(3-(2-(Cyclohexylsulfonyl)benzyl)-5-fluoro-2-methyl-indol-1-yl)-acetic acid;
2-(5-Fluoro-2-methyl-3-(2-(piperidin-1-ylsulfonyl)benzyl)-indol-1-yl)-acetic acid;
2-(3-(2-(Cyclopentylsulfonyl)benzyl)-5-fluoro-2-methyl-indol-1-yl)-acetic acid;
2-(5-Fluoro-2-methyl-3-(3-(piperidin-1-ylsulfonyl)benzyl)-indol-1-yl)-acetic acid;
2-(5-Fluoro-2-methyl-3-(2-(pyrrolidin-1-ylsulfonyl)benzyl)-indol-1-yl)-acetic acid;
2-(3-(4-(Cyclohexylsulfonyl)benzyl)-5-fluoro-2-methyl-indol-1-yl)-acetic acid;
2-(3-(4-(Cyclopentylsulfonyl)benzyl)-5-fluoro-2-methyl-indol-1-yl)-acetic acid;
2-(3-(2-(Cyclobutylsulfonyl)benzyl)-5-fluoro-2-methyl-indol-1-yl)-acetic acid;
2-(5-Fluoro-2-methyl-3-(3-(pyrrolidin-1-ylsulfonyl)benzyl)-indol-1-yl)-acetic acid;
2-(5-Fluoro-2-methyl-3-(4-(piperidin-1-ylsulfonyl)benzyl)-indol-1-yl)-acetic acid;
[5-Fluoro-2-methyl-3-(2-phenoxybenzyl)-indol-1-yl]-acetic acid;
[5-Fluoro-2-methyl-3-(2-(4-methoxyphenoxy)benzyl)-indol-1-yl]-acetic acid;
[5-Fluoro-2-methyl-3-(2-(4-methylphenoxy)benzyl)-indol-1-yl]-acetic acid;
[5-Fluoro-2-methyl-3-(2-(2,4-dichlorophenoxy)benzyl)-indol-1-yl]-acetic acid;
[5-Fluoro-2-methyl-3-(2-(4-fluorophenoxy)benzyl)-indol-1-yl]-acetic acid;
[5-Fluoro-2-methyl-3-(2-(3,4-difluorophenoxy)benzyl)-indol-1-yl]-acetic acid;
[5-Fluoro-2-methyl-3-(2-(4-cyanophenoxy)benzyl)-indol-1-yl]-acetic acid;
[5-Fluoro-2-methyl-3-(2-(4-chlorophenoxy)benzyl)-indol-1-yl]-acetic acid;
[5-Fluoro-2-methyl-3-(2-(2-cyanophenoxy)benzyl)-indol-1-yl]-acetic acid;
(5-Fluoro-2-methyl-3-{[2-(4-methylphenoxy)pyridin-3-yl]methyl}indol-1-yl)-acetic acid;
{5-Fluoro-3-[(3-methanesulfonylnaphthalen-2-yl)methyl]-2-methylindol-1-yl}-acetic acid;
{5-Fluoro-3-[(1-methanesulfonylnaphthalen-2-yl)methyl]-2-methylindol-1-yl}-acetic acid;
{5-Fluoro-3-[(6-methanesulfonylnaphthalen-2-yl)methyl]-2-methylindol-1-yl}-acetic acid;
[5-Fluoro-2-methyl-3-(quinolin-3-ylmethyl)indol-1-yl]-acetic acid;
[5-Fluoro-2-methyl-3-(quinoxalin-6-ylmethyl)indol-1-yl]-acetic acid;
[5-Fluoro-2-methyl-3-(quinolin-7-ylmethyl)indol-1-yl]-acetic acid;
{5-Fluoro-3-[(6-methanesulfonylquinolin-2-yl)methyl]-2-methylindol-1-yl}-acetic acid;
{5-Fluoro-3-[(4-methanesulfonylquinolin-2-yl)methyl]-2-methylindol-1-yl}-acetic acid;
(5-Fluoro-2-methyl-3-{pyrazolo[1,5-a]pyridin-3-ylmethyl}indol-1-yl)-acetic acid;
(5-Fluoro-3-{imidazo[1,2-a]pyridin-2-ylmethyl}-2-methylindol-1-yl)-acetic acid;
(5-Fluoro-2-methyl-3-{[2-(methylsulfanyl)phenyl]methyl}indol-1-yl)-acetic acid;
(5-Fluoro-2-methyl-3-{[3-(methylsulfanyl)phenyl]methyl}indol-1-yl)-acetic acid;
(5-Fluoro-2-methyl-3-{[4-(ethylsulfanyl)phenyl]methyl}indol-1-yl)-acetic acid;
(3-{[4-(Ethylsulfanyl)phenyl]methyl}-5-fluoro-2-methylindol-1-yl)-acetic acid;
(5-Fluoro-2-methyl-3-([4-(n-propylsulfanyl)phenyl]methyl)indol-1-yl)-acetic acid;
(5-Fluoro-2-methyl-3-{[4-(i-propylsulfanyl)phenyl]methyl}indol-1-yl)-acetic acid;
(5-Fluoro-2-methyl-3-{[4-(t-butylsulfanyl)phenyl]methyl}indol-1-yl)-acetic acid;
(5-Fluoro-2-methyl-3-([4-(pentan-3-ylsulfanyl)phenyl]methyl)indol-1-yl)-acetic acid;
[3-({4-[(Cyclopropylmethyl)sulfanyl]phenyl}methyl)-5-fluoro-2-methylindol-1-yl]-acetic acid;
{3-[(4,4-Dimethyl-2,3-dihydro-1-benzothiopyran-6-yl)methyl]-5-fluoro-2-methylindol-1-yl}-acetic acid;
(3-{[2-(Ethanesulfonyl)phenyl]methyl}-5-fluoro-2-methylindol-1-yl)-acetic acid;
(5-Fluoro-2-methyl-3-{[2-(propane-1-sulfonyl)phenyl]methyl}indol-1-yl)-acetic acid;
(5-Fluoro-2-methyl-3-{[2-(propane-2-sulfonyl)phenyl]methyl}indol-1-yl)-acetic acid;
(3-{[2-(Butane-1-sulfonyl)phenyl]methyl}-5-fluoro-2-methylindol-1-yl)-acetic acid;
(3-{[2-(Butane-2-sulfonyl)phenyl]methyl}-5-fluoro-2-methylindol-1-yl)-acetic acid;
(5-Fluoro-2-methyl-3-{[2-(2-methylpropane-2-sulfonyl)phenyl]methyl}indol-1-yl)-acetic acid;
(5-Fluoro-2-methyl-3-([2-(pentane-1-sulfonyl)phenyl]methyl)indol-1-yl)-acetic acid;
(3-{[2-(Cyclopropylmethane)sulfonylphenyl]methyl}-5-fluoro-2-methylindol-1-yl)-acetic acid;
(5-Fluoro-2-methyl-3-{[2-(propylsulfamoyl)phenyl]methyl}indol-1-yl)-acetic acid;
(3-{[2-(Butylsulfamoyl)phenyl]methyl}-5-fluoro-2-methylindol-1-yl)-acetic acid;
(5-Fluoro-2-methyl-3-{[3-(propylsulfamoyl)phenyl]methyl}indol-1-yl)-acetic acid;
(3-{[3-(Butylsulfamoyl)phenyl]methyl}-5-fluoro-2-methylindol-1-yl)-acetic acid;
(5-Fluoro-2-methyl-3-{[4-(trifluoromethane)sulfonylphenyl]methyl}indol-1-yl)-acetic acid;
(3-{[4-(Ethanesulfonyl)phenyl]methyl}-5-fluoro-2-methylindol-1-yl)-acetic acid;
(5-Fluoro-2-methyl-3-{[4-(propane-1-sulfonyl)phenyl]methyl}indol-1-yl)-acetic acid;
(5-Fluoro-2-methyl-3-{[4-(propane-2-sulfonyl)phenyl]methyl}indol-1-yl)-acetic acid;
(3-{[4-(Butane-1-sulfonyl)phenyl]methyl}-5-fluoro-2-methylindol-1-yl)-acetic acid;
(5-Fluoro-2-methyl-3-{[4-(2-methylpropane-2-sulfonyl)phenyl]methyl}indol-1-yl)-acetic acid;
(5-Fluoro-2-methyl-3-{[4-(pentane-1-sulfonyl)phenyl]methyl}indol-1-yl)-acetic acid;
(5-Fluoro-2-methyl-3-{[4-(pentan-3-ylsulfonyl)phenyl]methyl}indol-1-yl)-acetic acid;
[3-({4-[(Cyclopropylmethyl)sulfonyl]phenyl}methyl)-5-fluoro-2-methylindol-1-yl]-acetic acid;
(5-Fluoro-2-methyl-3-{[4-(propylsulfamoyl)phenyl]methyl}indol-1-yl)-acetic acid;
(3-{[4-(Butylsulfamoyl)phenyl]methyl}-5-fluoro-2-methylindol-1-yl)-acetic acid;
(5-Fluoro-2-methyl-3-{[4-(trifluoromethoxy)phenyl]methyl}indol-1-yl)-acetic acid;
(5-Fluoro-3-{[4-methanesulfonyl-3-(trifluoromethyl)phenyl]methyl}-2-methylindol-1-yl)-acetic acid;
(5-Fluoro-3-{[4-methanesulfonyl-3-(trifluoromethoxy)phenyl]methyl}-2-methylindol-1-yl)-acetic acid;
{5-Fluoro-3-[(5-methanesulfonylthiophen-2-yl)methyl]-2-methylindol-1-yl}-acetic acid;
{3-[(4,4-dimethyl-1,1-dioxo-2,3-dihydro-1λ⁶-benzothiopyran-6-yl)methyl]-5-fluoro-2-methylindol-1-yl}-acetic acid;
[3-({1-[(4-Chlorobenzene)sulfonyl]pyrrol-2-yl}methyl)-5-fluoro-2-methylindol-1-yl]-acetic acid;
[5-Fluoro-3-({1-[(4-fluorobenzene)sulfonyl]pyrrol-2-yl}methyl)-2-methylindol-1-yl]-acetic acid;
[5-Fluoro-3-({1-[(4-methoxybenzene)sulfonyl]pyrrol-2-yl}methyl)-2-methylindol-1-yl]-acetic acid;
{3-[1-(2,4-Dichloro-benzenesulfonyl)pyrrol-2-ylmethyl]-5-fluoro-2-methyl-indol-1-yl}-acetic acid;
[5-Fluoro-3-({1-[(4-methanesulfonylbenzene)sulfonyl]pyrrol-2-yl}methyl)-2-methylindol-1-yl]-acetic acid;
{5-Fluoro-2-methyl-3-[(2-phenylphenyl)methyl]indol-1-yl}-acetic acid;
(3-{[1-(Benzenesulfonyl)indol-2-yl]methyl}-5-fluoro-2-methylindol-1-yl)-acetic acid;
(3-{[2-(4-Chlorophenyl)phenyl]methyl}-5-fluoro-2-methylindol-1-yl)-acetic acid;
(5-Fluoro-2-methyl-3-{[2-(4-methylphenyl)phenyl]methyl}indol-1-yl)-acetic acid;
{5-Fluoro-2-methyl-3-[(3-phenoxyphenyl)methyl]indol-1-yl}-acetic acid;
[5-Fluoro-3-({4-[(4-fluorophenyl)carbonyl]-1-methylpyrrol-2-yl}methyl)-2-methylindol-1-yl]-acetic acid;
(5-Fluoro-2-methyl-3-[(6-([3-(trifluoromethyl)phenyl]methylpyridin-3-yl)methyl]indol-1-yl)-acetic acid;
5-Fluoro-2-methyl-3-[(3-phenoxythiophen-2-yl)methyl]indol-1-yl)-acetic acid;
(3-{[2-(Benzenesulfonyl)-1,3-thiazol-5-yl]methyl}-5-fluoro-2-methylindol-1-yl)-acetic acid;
{3-[(1-Benzylpyrazol-4-yl)methyl]-5-fluoro-2-methylindol-1-yl}-acetic acid;
(3-{[5-(4-Chlorophenoxy)-1-methyl-3-(trifluoromethyl)pyrazol-4-yl]methyl}-5-fluoro-2-methylindol-1-yl)-acetic acid;
[3-({5-[(4-Chlorobenzene)sulfonyl]furan-2-yl}methyl)-5-fluoro-2-methylindol-1-yl]-acetic acid;
[3-({5-[(4-Chlorobenzene)sulfonyl]thiophen-2-yl}methyl)-5-fluoro-2-methylindol-1-yl]-acetic acid;
[3-({3-[(4-Chlorobenzene)sulfonyl]thiophen-2-yl}methyl)-5-fluoro-2-methylindol-1-yl]-acetic acid;
{3-[(2-Benzylphenyl)methyl]-5-fluoro-2-methylindol-1-yl}-acetic acid;
or the C₁-C₆ alkyl, aryl, (CH₂)ₘOC(═O)C₁-C₆alkyl, ((CH₂)ₘO)ₙCH₂CH₂X, (CH₂)ₘN(R⁷)₂, or CH((CH₂)ₘO(C═O)R⁸)₂ esters of any of the above; wherein
m is 1 or 2;
n is 1-4;
X is OR⁷ or N(R⁷)₂;
R⁷ is hydrogen or methyl;
R⁸ is C₁-C₁₈ alkyl.

15. The method of claim 1, wherein the compound of Formula (I) is administered in combination with one or more additional agent which is of use in the treatment of an eosinophilic or atopic disease or condition.

16. The method of claim 15, wherein the additional agent is selected from the group consisting of montelukast; β adrenoreceptor agonists such as metaproterenol, isoproterenol, isoprenaline, albuterol, salbutamol, formoterol, salmeterol, terbutaline, orciprenaline, bitolterol mesylate, pirbuterol, and indacaterol; inhaled corticosteroids such as prednisone, prednisolone, flunisolide, triamcinolone acetonide, beclomethasone dipropionate, budesonide, fluticasone propionate, fluticasone furoate, mometasone furoate, and ciclesonide; muscarinic receptor antagonists such as ipratropium and tiotropium; anti-IL-5 antibodies such as mepolizumab and reslizumab; anti-IgE antibodies such as omalizumab; and agents which block the actions of IL-4 and/or IL-13 such as the IL-4 mutein pitrakinra and anti-IL-13 receptor α antibodies such as AMG-317 and anti-IL-13 antibodies.

17. The method of claim 1, wherein the compound of Formula (I) is administered via the oral, nasal, bronchial, or topical route.

18. The method of claim 1, wherein the eosinophilic disease or condition is selected from the group consisting of eosinophilic asthma, atopic asthma, uncontrolled asthma, eosinophilic chronic obstructive pulmonary disease, eosinophilic nasal polyps, eosinophilic oesophagitis, eosinophilic atopic dermatitis, eosinophilic allergic conjunctivitis, allergic rhinitis, and Churg Strauss sydrome.

19. The method of claim 1, wherein the degree of airway cosinophilia is measured based on baseline blood eosinophil count.

20. The method of claim 19, wherein the degree of airway eosinophlia is determined to be high when the baseline blood eosinophil count is greater than 200 cells/μL.

21. The method of claim 19, wherein the degree of airway eosinophilia is determined to be high when the baseline blood eosinophil count is greater than 250 cells/μL.

22. The method of claim 1, wherein the degree of airway eosinophilia is measured based on baseline sputum eosinophil percentage.

23. The method of claim 22, wherein the degree of airway eosinophilia is determined to be high when the baseline sputum eosinophil percentage is greater than 2%.

24. The method of claim 22, wherein the degree of airway eosinophilia is determined to be high when the baseline sputum eosinophil percentage is greater than 2.5%.

25. The method of claim 1, wherein the degree of airway eosinophilia is measured based on a baseline ACQ score.

26. The method of claim 25, wherein the degree of airway eosinophilia is determined to be high when the baseline ACQ score is at least 1.5.

27. The method of claim 25, wherein the degree of airway eosinophilia is determined to be high when the baseline ACQ score is at least 2.0.

28. The method of claim 1, wherein the degree of airway eosinophilia is measured based on baseline ACQ score and baseline blood eosinophil count.

29. The method of claim 28, wherein the degree of airway eosinophilia is determined to be high when the baseline ACQ score is at least 1.5 and the baseline blood eosinophil count is greater than 200 cells/μL.

30. The method of claim 28, wherein the degree of airway eosinophilia is determined to be high when the baseline ACQ score is at least 1.5 and the baseline blood eosinophil count is greater than 250 cells/μL.

31. The method of claim 1, wherein the subject also has a high atopic status.

32. The method of claim 31, wherein the atopic status is measured based on a skin prick test.

33. The method of claim 31, wherein and the degree of airway eosinophilia is measured based on baseline ACQ score and baseline blood eosinophil count.

34. The method of claim 33, wherein the atopic status is determined to be high when the skin prick test is positive and the degree of airway eosinophilia is determined to be high when the baseline ACQ score is at least 1.5 and the baseline blood eosinophil count is greater than 200 cells/μL.

35. The method of claim 1, wherein the subject is less than or equal to 50 years of age.

36. The method of claim 1, wherein the subject is less than or equal to 30 years of age.

37. The method of claim 1, wherein the subject is less than or equal to 30 years of age at onset of the eosinophilic disease or condition.

38. The method of claim 1, wherein the subject is between about 0 and about 10 years of age at onset of the eosinophilic disease or condition.

39. The method of claim 1, wherein the subject is between about 11 and about 20 years of age at onset of the eosinophilic disease or condition.

40. A method of treating an eosinophilic disease or condition in a subject with a high atopic status, comprising:
(a) determining the atopic status of the subject;
(b) administering a compound of Formula (I) to said subject if the atopic status in (a) is at or above a level determined to be high:
wherein the compound of Formula (I): 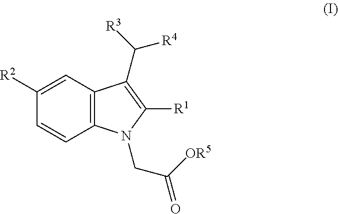
wherein
R¹ is C₁-C₆ alkyl;
R² is halogen; and
R³ is aryl or heteroaryl optionally substituted with one or more substituents selected from halo, OH, CN, R⁶, COR⁶, CH₂R⁶, OR⁶, SR⁶, SO₂R⁶ or SO₂YR⁶;
R⁶ is C₁-C₆ alkyl, C₃-C₈ cycloalkyl, heterocyclyl, aryl or heteroaryl, any of which may optionally be substituted with one or more substituents selected from halo, OH, CN, NO₂, C₁-C₆ alkyl or O(C₁-C₆ alkyl); and
Y is NH or a straight or branched C₁-C₄ alkylene chain;
R⁴ is H or C₁-C₄ alkyl;
R⁵ is hydrogen, C₁-C₆ alkyl, aryl, (CH₂)ₘOC(═O)C₁-C₆alkyl, ((CH₂)ₘO)ₙCH₂CH₂X, (CH—₂)ₘN(R⁷), or CH((CH₂)ₘO(C═O)R⁸)₂;
m is 1 or 2;
n is 1-4;
X is OR⁷ or N(R⁷)₂;
R⁷ is hydrogen or methyl;
R⁸ is C₁-C₁₈ alkyl;
or a pharmaceutically acceptable salt, hydrate, solvate, or complex thereof.

41. The method of claim 40, wherein the atopic status is measured based on serum IgE levels.

42. The method of claim 40, wherein the atopic status is determined to be high when the serum IgE level is greater than 100 units/mL.

43. The method of claim 40, wherein the atopic status is determined to be high when the serum IgE level is greater than 200 units/mL.
